(19) United States
(12) Patent Application Publication
(10) Pub. No.: US 2007/0106119 A1
(43) Pub. Date: May 10, 2007

(54) ENDOSCOPE

(76) Inventors: Yasuo HIRATA, Tokyo (JP); Shinji FUJIKAWA, Tokyo (JP)

Correspondence Address: OSTROLENK FABER GERB & SOFFEN, 1180 AVENUE OF THE AMERICAS, NEW YORK, NY, 100368403, US

(21) Appl. No.: 11/617,568

(22) Filed: Dec. 28, 2006

(30) Foreign Application Priority Data
Jun. 29, 2005 (JP) 2005-190715

(51) Int. Cl. A61B 1/06

(52) U.S. Cl. 600/179; 600/129; 600/175

(57) ABSTRACT

An endoscope includes an insertion portion formed in an elongated cylindrical shape; and a cylindrical shaped image pickup adaptor, the pickup adaptor comprising, an electric device arranged on the distal end side thereof, and a proximal end side of the image pickup adaptor being attached to and removed from the distal end side of the insertion portion, wherein a pair of adaptor side electrode terminals conducting with the electric device are arranged on the image pickup adaptor, and a pair of insertion portion side electrode terminals that respectively come into contact with and are electrically connected to the pair of adaptor side electrode terminals, are arranged on the insertion portion so as to respectively face, and be able to come in contact with, the pair of adaptor side electrode terminals, when the image pickup adaptor is attached to the insertion portion.

### BACKGROUND OF THE INVENTION

1. Field of the Invention

The present invention relates to an endoscope for industrial and medical purposes to be used with an image pickup adaptor that is attached to the endoscope and comprising electric devices, such as a light emitting diode, on a distal end of an elongated insertion portion to be inserted into a body lumen.

2. Description of Related Art

Generally, as an endoscope to be used for industrial and medical purposes, an endoscope has been proposed that is used with an image pickup adaptor attached to a distal end of an insertion portion of the endoscope and comprising a light emitting diode (hereinafter, referred to as LED) as an illumination light source for illuminating a subject to be observed inside a body lumen to perform observation and image pickup easily.

Since the image pickup adaptor and the insertion portion are separated from each other in such an endoscope, an endoscope has been proposed in which the image pickup adaptor and the insertion portion are conducted by bringing an insertion portion side electrode terminal into contact with a plate spring having an appropriate degree of biasing force in order to ensure a reliable connection between the image pickup adaptor and the insertion portion (for example, Japanese Unexamined Patent Application, First Publication No. 2005-27851 (FIG. 6)). In this case, the plate spring and the insertion portion side electrode terminal are formed so that a condition of their contact sufficient to ensure conduction is maintained

Meanwhile, in the case where electric power is to be supplied to an electric device such as a LED arranged within the image pickup adaptor, electrode terminals for connection are arranged on both the image pickup adaptor and the insertion portion. In this case, in order to reliably supply electric power to the electric device, both of the electrode terminals need to be reliably brought in contact with each other. Therefore, an endoscope having a structure for establishing contact by bringing an elastic spring in contact with a hard electrode terminal or by using male/female connectors has been proposed (Japanese Unexamined Patent Application, First Publication No. H10-328131 (FIG. 5)).

Incidentally, in the endoscope mentioned above, following expansion in its application, reduction in an outer diameter of the insertion portion from approximately 12 to 13 mm, used in a general endoscope apparatus, to approximately 0.5 mm, used for inserting into a blood vessel, has been achieved.

Against such a background, the image pickup adaptor and the insertion portion are required to have an attachment/removal structure that enables reliable electrical connection, and a connection structure that does not impede a reduction in the diameter of the insertion portion.

### SUMMARY OF THE INVENTION

The endoscope according to the present invention is characterized in that it is an endoscope comprising: an insertion portion formed in an elongated cylindrical shape; a cylindrical shaped image pickup adaptor, the pickup adaptor comprising, an electric device arranged on the distal end side thereof; and a proximal end side of the image pickup adaptor being attached to and removed from the distal end side of the insertion portion, wherein a pair of adaptor side electrode terminals continuous with the electric device are lined up in a direction different to the direction in which the image pickup adaptor and the insertion portion are attached and detached to and from each other, and are arranged on the image pickup adaptor facing in a direction orthogonal to the above direction, and a pair of insertion portion side electrode terminals that respectively come into contact with and are electrically connected to the pair of adaptor side electrode terminals, are arranged on the insertion portion so as to respectively face, and be able to come in contact with, the pair of adaptor side electrode terminals, when the image pickup adaptor is attached to the insertion portion.

Preferably, an endoscope according to the present invention is the aforementioned endoscope, wherein the pair of adaptor side electrode terminals and the pair of insertion portion side electrode terminals are arranged in pairs in a direction different from the direction in which the image pickup adaptor and the insertion portion are attached to and detached from each other.

Preferably, an endoscope according to the present invention is the aforementioned endoscope, wherein the pair of adaptor side electrode terminals and the pair of insertion portion side electrode terminals are arranged so as to face each other in a direction orthogonal to the direction in which the image pickup adaptor and the insertion portion are attached to and detached from each other.

Preferably, an endoscope according to the present invention is the aforementioned endoscope, wherein the pair of adaptor side electrode terminals and the pair of insertion portion side electrode terminals are arranged so as to face each other in a direction inclined at a constant angle to the direction in which the image pickup adaptor and the insertion portion are attached to detached from each other.

Preferably, an endoscope according to the present invention is the aforementioned endoscope, further comprising an aligning section which adjusts the orientation of the image pickup adaptor to the direction in which the pair of adaptor side electrode terminals and the pair of insertion portion side electrode terminals face each other, when the image pickup adaptor is attached to the insertion portion.

Preferably, an endoscope according to the present invention is the aforementioned endoscope, further comprising a biasing device which adjusts a pressing force of the pair of adaptor side electrode terminals and the pair of insertion portion side electrode terminals, when the image pickup adaptor is attached to the insertion portion.

Preferably, an endoscope according to the present invention is the aforementioned endoscope, wherein at least one of the pair of insertion portion side electrode terminals and the pair of adaptor side electrode terminals is formed from an elastic member.

Preferably, an endoscope according to the present invention is the aforementioned endoscope, wherein the pair of insertion portion side electrode terminals and the pair of adaptor side electrode terminals comprise facing surfaces that face each other.

### BRIEF DESCRIPTION OF THE DRAWINGS

FIG. 1A is a perspective view showing an endoscope according to a first embodiment of the present invention when disassembled.

FIG. 1B is a perspective view showing the endoscope according to the first embodiment of the present invention when assembled and housed.

FIG. 2A is an exploded perspective view showing the endoscope according to the first embodiment of the present invention.

FIGS. 2B and 2C are perspective views showing an adaptor of the endoscope according to the first embodiment of the present invention.

FIG. 3A is a longitudinal sectional view showing the adaptor of the endoscope according to the first embodiment of the present invention.

FIG. 3B is a longitudinal sectional view showing a distal end of an insertion portion of the endoscope according to the first embodiment of the present invention

FIG. 4 is a longitudinal sectional view showing an adaptor and a distal end of an insertion portion of an endoscope according to a second embodiment of the present invention.

FIG. 5A is an exploded perspective view showing an endoscope according to a third embodiment of the present invention.

FIG. 5B is a longitudinal sectional view showing an adaptor of the endoscope according to the third embodiment of the present invention.

FIG. 5C is a front view seen from a proximal end side showing the adaptor of the endoscope according to the third embodiment of the present invention.

FIG. 6A is an exploded perspective view showing an endoscope according to a fourth embodiment of the present invention.

FIG, 6B is a longitudinal sectional view showing an adaptor of the endoscope according to the fourth embodiment of the present invention.

FIG. 6C is a longitudinal sectional view showing a distal end of an insertion portion of the endoscope according to the fourth embodiment of the present invention.

FIG. 7A is an exploded perspective view showing a principal portion of an adaptor of an endoscope according to a fifth embodiment of the present invention.

FIG. 7B is a longitudinal sectional view showing the adaptor and a distal end of an insertion portion of the endoscope according to the fifth embodiment of the present invention.

FIG. 8A is a longitudinal sectional view showing respectively an adaptor and a distal end of an insertion portion of an endoscope according to a sixth embodiment of the present invention.

FIG. 8B is a perspective view showing a principal portion of the distal end of the insertion portion of the endoscope according to the sixth embodiment of the present invention.

FIG. 8C is a longitudinal sectional view showing the distal end of the insertion portion of the endoscope according to the sixth embodiment of the present invention.

FIG. 9A is a longitudinal sectional view showing respectively an adaptor and a distal end of an insertion portion of an endoscope according to a seventh embodiment of the present invention.

FIG. 9B is a perspective view showing a principal portion of the adaptor of the endoscope according to the seventh embodiment of the present invention,

FIG. 10A is a longitudinal sectional view showing respectively an adaptor and a distal end of an insertion portion of an endoscope according to an eighth embodiment of the present invention.

FIG. 10B is a perspective view showing a principal portion of the adaptor of the endoscope according to the eighth embodiment of the present invention.

FIG. 11A is a perspective view showing a modification of a distal end of an insertion portion of the endoscope according to the eighth embodiment of the present invention

FIG. 11B is a perspective view showing a principal portion of a modification of the adaptor of the endoscope according to the eighth embodiment of the present invention.

FIG. 12A is an exploded perspective view showing an adaptor of an endoscope according to a ninth embodiment of the present invention.

FIG. 12B is a longitudinal sectional view showing the adaptor of the endoscope according to the ninth embodiment of the present invention.

FIG. 13A is an exploded perspective view showing a modification of the adaptor of the endoscope according to the ninth embodiment of the present invention.

FIG. 13B is a longitudinal sectional view showing a modification of the adaptor of the endoscope according to the ninth embodiment of the present invention.

FIG. 14A is an exploded perspective view showing an endoscope according to a tenth embodiment of the present invention.

FIG. 14B is a longitudinal sectional view showing a distal end of an insertion portion according to the tenth embodiment of the present invention.

FIG. 14C is a longitudinal sectional view showing an adaptor according to the tenth embodiment of the present invention.

FIG. 15A is a longitudinal sectional view showing respectively an adaptor and a distal end of an insertion portion of an endoscope according to an eleventh embodiment of the present invention.

FIG. 15B is a development view showing a flexible substrate on an insertion portion side of the distal end of the insertion portion of the endoscope according to the eleventh embodiment of the present invention.

FIG. 15C is a perspective view showing a flexible substrate on an insertion portion side of a distal end of an insertion portion of the endoscope according to the eleventh embodiment of the present invention.

FIG. 15D is a peeve view showing the distal end of the insertion portion of the endoscope according to the eleventh embodiment of the present invention.

FIG. 15E is a side view showing an insert flexible substrate on the insertion portion side of the distal end of the insertion portion of the endoscope according to the eleventh embodiment of the present invention.

FIG. 15F is a diagram showing an insert flexible substrate on the insertion portion side of the distal end of the insertion portion of the endoscope according to the eleventh embodiment of the present invention.

FIG. 16A is a longitudinal sectional view showing respectively an adaptor and a distal end of an insertion portion of an endoscope according to a twelfth embodiment of the present invention.

FIG. 16B is a perspective view showing the adaptor of the endoscope according to the twelfth embodiment of the present invention.

FIG. 16C is a perspective view showing a flexible substrate of the adaptor of the endoscope according to the twelfth embodiment of the present invention.

FIG. 16D is a side view showing a flexible substrate on which adaptor side electric contact points are arranged of the endoscope according to the twelfth embodiment of the present invention

FIG. 16E and FIG. 16F are perspective views showing the distal end of the insertion portion of the endoscope according to the twelfth embodiment of the present invention.

FIG. 16G is an enlarged sectional view showing a principal portion of the distal end of the insertion portion of the endoscope according to the twelfth embodiment of the present invention.

FIG. 16H is an enlarged perspective view showing a principal portion of the distal end of the insertion portion of the endoscope according to the twelfth embodiment of the present invention.

FIG. 17A is a longitudinal sectional view showing respectively an adaptor and a distal end of an insertion portion of an endoscope according to a thirteenth embodiment of the present invention.

FIG. 17B is a perspective view showing a principal portion of the distal end of the insertion portion of the endoscope according to the thirteenth embodiment of the present invention.

FIG. 17C is an explanatory diagram showing a balloon of the distal end of the insertion portion of the endoscope according to the thirteenth embodiment of the present invention when inflated.

FIG. 17D is a sectional view showing the balloon of the distal end of the insertion portion of the endoscope according to the thirteenth embodiment of the present invention when inflated.

FIG. 18A is a perspective view showing respectively an adaptor and a distal end of an insertion portion of an endoscope according to a fourteenth embodiment of the present invention.

FIG. 18B is a front view seen from a proximal end side showing the adaptor of the endoscope according to the fourteenth embodiment of the present invention.

FIG. 18C is a front view seen from a distal end side showing the distal end of the insertion portion of the endoscope according to the fourteenth embodiment of the present invention.

FIG. 19A is a perspective view showing a flexible substrate provided for the adaptor of the endoscope according to the third embodiment of the present invention.

FIG. 19B is a perspective view showing a flexible substrate provided for the adaptor of an endoscope according to another embodiment of the present invention.

FIG. 20A is an exploded perspective view showing an endoscope according to another embodiment of the present invention.

FIG. 20B is a perspective view showing the adaptor of an endoscope according to another embodiment of the present invention.

FIG. 20C is a perspective view showing a distal end of an insertion portion of an endoscope.

FIG. 21 is a longitudinal sectional view showing the adaptor and the distal end of the insertion portion of the endoscope shown in FIG. 20.

### DETAILED DESCRIPTION OF THE INVENTION

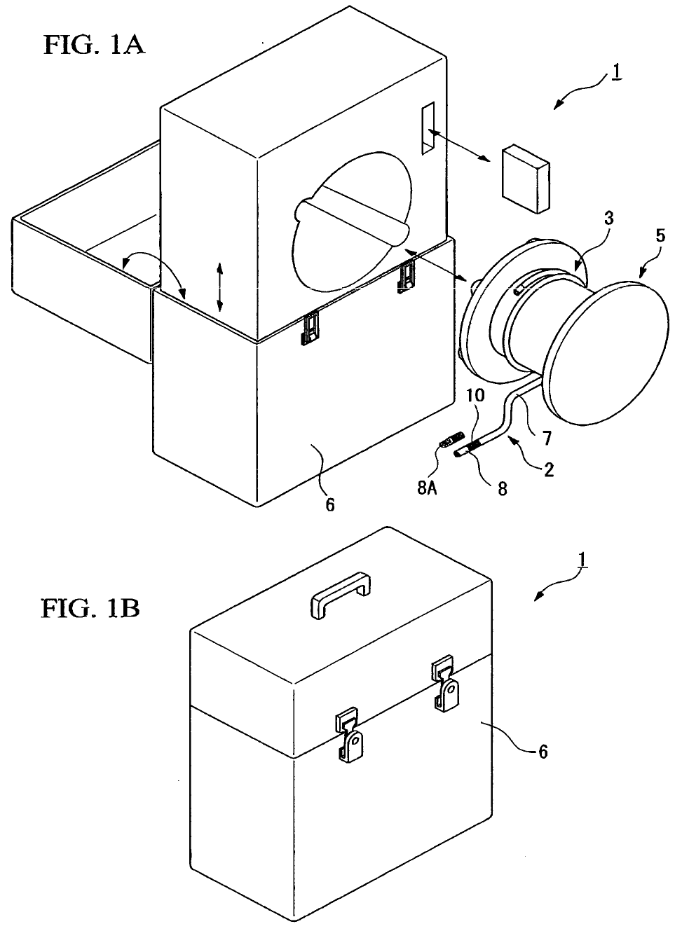

A first embodiment according to the present invention is described, with reference to FIG. 1A to FIG. 3B.

An endoscope 1 according to the present embodiment comprises, as the principal components thereof, an endoscope main body 3 comprising an elongated insertion portion 2; and a drum section 5 around which the insertion portion 2 of the endoscope main body 3 is wound to house it.

The endoscope main body 3 is housed inside a casing 6 that accommodates the drum section 5 allowing free rotation, and stored and transported therein.

The insertion portion 2 of the endoscope main body 3 comprises an elongated flexible-tube section 7 to be inserted into a lumen of a subject to be observed.

An adaptor (image pick up adaptor) 8 on which an electric device such as an illumination light souse is arranged, and a main body distal end section 10 which will be described later, to and from which another adaptor 8A is attached and detached, are arranged on a distal end of the flexible tube section 7.

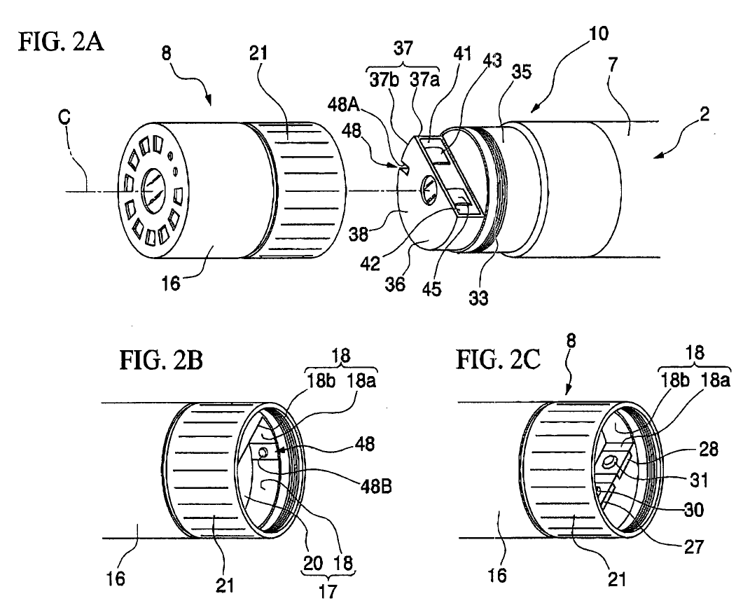

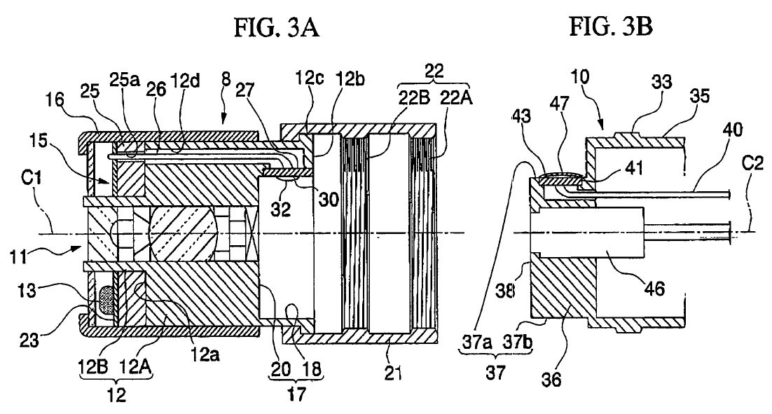

The adaptor 8 is a so-called direct view type adaptor in which an objective lens 11 is arranged along a central axis C1 direction of the adaptor 8 as shown in FIG. 2A to FIG. 2C and FIG. 3A and FIG. 3B. The objective lens 11 is housed inside a lens cylinder section 12 formed in a substantially cylindrical shape, and it is housed in an outside member 16 formed in a cylindrical shape together with a LED unit (electrical device) 15 on which a plurality of LED chips 13 are arranged.

The lens cylinder section 12 comprises a thick cylindrically shaped base section 12A and a cylinder wall 12B that is arranged so as to project from a distal end surface 12a of the base section 12A, and whose outer surface has a diameter smaller than that the outer surface of the base section 12A and whose inner surface is formed so as to have an inner diameter substantially equal to the inner diameter of the base section 12A. A depressed section 17 having a substantially D shaped cross-sectional shape is formed in a proximal end surface 12b of the base section 12A. The depressed section 17 comprises a side surface section 18 comprising a first plane surface 18a that includes one portion of a line segment connecting two points on an outer circumferential surface of the base section 12A, and of a first curved surface 18b that includes one portion of an arc along the outer circumferential surface, and a base section 20.

A flange section 12c that projects outward in a radial direction is formed on a periphery section of the proximal end surface 12b of the base section 12A. A connection ring 21 that is engaged with the lens cylinder section 12 so as to be freely rotatable around the central axis C1, is arranged on this flange section 12c. A female screw section 22 comprising a first female screw section 22A and a second female screw section 22B arranged along the central axis C1, is formed on an inner surface of the connection ring 21.

The LED unit 15 comprises a flexible substrate 23 formed from soft material in a disc shape on which the plurality of LED chips 13 are mounted; and a thick disc shape aluminum substrate 25 that is formed from a metal material such as aluminum and supports the flexible substrate 23 within the lens cylinder 12. The LED unit 15 is arranged fitted to the cylinder wall 12B of the lens cylinder section 12.

An insert hole 25a and a through hole 12d through which a pair of electric wires (only the anode side is shown in the diagram) 26, one end of which is electrically connected to the flexible substrate 23, is inserted substantially in parallel with the direction of the central axis C1, are arranged in the aluminum substrate 25 and the base section 12A of the lens cylinder section 12, respectively.

A pair of adaptor side terminal substrates 27 and 28 is respectively connected to the other end of the pair of the electric wires, and a pair of adaptor side electrode terminals 30 and 31 is arranged on the pair of adaptor side terminal substrates 27 and 28, respectively.

The pair of adaptor side electrode terminals 30 and 31 is respectively arranged in a pair on the first plane surface 18a of the depressed section 17 of the lens cylinder section 12 via the pair of adaptor side terminal substrates 27 and 28, in a direction orthogonal to the direction in which the adaptor 8 and insertion portion 2 attach and detach. This pair of adaptor side electrode terminals 30 and 31 is respectively comprising an adaptor side facing surface (facing surface) 32 that faces inward in the radial direction as a top surface of each of the terminals.

The main body distal end section 10 comprises a cylinder section 35 formed in a cylindrical shape on which a male screw section 33 is formed to be screwed into the female screw section 22 of the connection ring 21. A prowling section 36 that is formed in a substantially D shaped cross-sectional shape and that fits into the depressed section 17 of the adaptor 8, is arranged on a distal end side of the cylinder section 35. The protruding section 36 comprises an insertion-portion-side side surface section 37 comprising a second plane surface 37a that faces the first plane surface 18a of the depressed section 17, and a second curved surface 37b that faces the first curved surface 18b of the depressed section; and a distal end surface 38.

A pair of insertion portion side terminal substrates 41 and 42 connected to an electric wire 40 (not shown) that extends from a power supply (such as a battery) in the drum section 5, are arranged on the second plane surface 37a of the protruding section 36 of the main body distal end section 10. A pair of insertion portion side electrode terminals 43 and 45 that come into contact respectively with the pair of adaptor side electrode terminals 30 and 31 to be electrically connected to the LED unit 15 when the insertion portion 2 is attached to the adaptor 8, are arranged on the pair of insertion portion side terminal substrates 41 and 42, respectively. Moreover, a CCD 46 that images an endoscopic subject via the objective lens 11, is arranged on a central axis C2 of the protruding section 36.

The pair of insertion portion side electrode terminals 43 and 45 comprises an insertion portion side facing surface (facing surface) 47 that faces inward in the radial direction as a top surface of each of the terminals, so as to face the pair of adaptor side electrode terminals 30 and 31 when the adaptor 8 is attached.

A distance between the central axis C2 and the insertion portion side facing surface 47 is substantially equal to the distance between the central axis C1 and the adaptor side facing surface 32, or, the distance to the insertion portion side facing surface 47 is slightly greater than that to the adaptor side facing surface 32, so that the inert section side facing surface 47 and the adaptor side facing surface 32 can contact with each other.

An aligning section 48 that adjusts the orientation of the adaptor 8 so that the adaptor side facing surface 32 and the insertion portion side facing surface 47 face each other when the adaptor 8 is attached to the insertion portion 2 with the central axis C1 and the central axis C2 aligned on a same axis, is arranged on the first curved surface 18b of the depressed section 17 and on the second curved surface 37b of the protruding section 36.

The aligning section 48 comprises a groove section 48A arranged on the second curved space 37b of the protruding section 36 and a protruding engaging section 48B arranged on the first curved surface 18b of the depressed section 17 so as to project inward in a radial direction to be engaged with the groove section. The respective terminal substrates are configured in pairs, as described for the pair of adaptor side terminal substrates 27 and 28 and the pair of insertion portion side terminal substrates 41 and 42, however, two terminals may be arranged on an integrated terminal substrate instead of on the separate terminal substrates.

Next, the effects of using the endoscope 1 according to the present embodiment are described.

First, the protruding section 36 is inserted into the depressed section 17 with the engaging section 48B of the depressed section 17 of the adaptor 8 engaged with the groove section 48A of the protruding section 36.

At this time, the adaptor side facing surfaces 32 of the pair of adaptor side electrode terminals 30 and 31 begin to come into contact with the insertion portion side facing surfaces 47 of the pair of insertion portion side electrode terminals 43 and 45, and the area of their contact increases as insertion gradually progresses.

The male screw section 33 is screwed into the female screw section 22 of the connection ring 21 of the adaptor 8 to bring the distal end surface 38 of the protruding section 36 into contact with a bottom section 20 of the depressed section 17 of the lens cylinder section 12. At this time, the area of contact between the adaptor side facing surface 32 and the insertion portion side facing surface 47 reaches a maximum. Specifically, the pair of insertion portion side electrode terminals 43 and 45 is pressed against the pair of adaptor side electrode terminals 30 and 31 and they are brought in a close contact with each other. Therefore, the pair of adaptor side electrode terminals 30 and 31 is continuous with the pair of insertion portion side electrode terminals 43 and 45 so that the adaptor 8 and the main body distal end section 10 are electrically connected.

In this way, a predetermined observation is performed by inserting the insertion portion 2 into a lumen, which is a subject to be observed (not shown).

When the observation is finished, the insertion portion 2 is withdrawn from the lumen, and the connection ring 21 and main body distal end section 10 are unscrewed. At this time, the adaptor side facing surfaces 32 of the pair of adaptor side electrode terminals 30 and 31 begin to separate from the insertion portion side facing surfaces 47 of the pair of insertion portion side electrode terminals 43 and 45, and the adaptor 8 and insertion portion 2 are electrically disconnected.

According to the endoscope 1, the pair of adaptor side electrode terminals 30 and 31 and the pair of insertion portion side electrode terminals 43 and 45 can be brought into surface-contact on their respective facing surfaces 32 and 47, and the area of their contact can be successively increased from the start to the end of attachment. Therefore, the adaptor 8 and the insertion portion 2 can be electrically connected more reliably than in the case of line contact.

Here, the pair of adaptor side electrode terminal 30 and 31 and the pair of insertion portion side electrode terminals 43 and 45 are arranged side by side in a direction orthogonal to the direction of attachment and removal, on the first plane surface 18a and the second plane surface 37a, which have a normal line of different direction from that of the attachment and removal of the adaptor 8. As a result, when attaching or detaching the adaptor 8, the pair of adaptor side electrode terminals 30 and 31 and the pair of insertion portion side electrode terminals 43 and 45 can be brought in contact at the same time, and wear of the terminals can be minimized for a reasonable number of times that the terminals come in contact.

Therefore, the pair of adaptor side electrode terminals 30 and 31 and the pair of insertion portion side electrode terminals 43 and 45 can be brought in contact with a reliable contact-force even in the case of small size, and a reduction in the size of the insertion portion 2 and the adaptor 8 can be achieved.

Furthermore, since the aligning section 48 is provided, by attaching the insertion portion 2 to the adaptor 8 while positioning by the aligning section 48, the respective facing surfaces 32 and 47 of the pair of adaptor side electrode terminals 30 and 31 and the pair of insertion portion side electrode terminals 43 and 45 can be easily and reliably brought into contact.

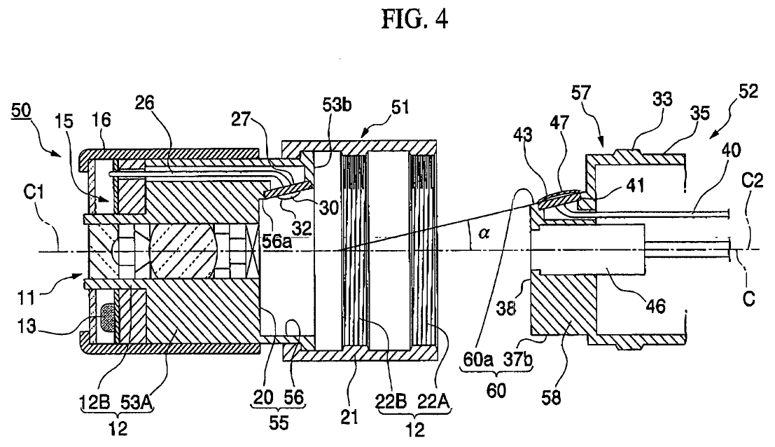

Next, a second embodiment is described, with reference to FIG. 4.

Components similar to those in the first embodiment described above are given same reference symbols, and descriptions thereof are omitted.

The second embodiment differs from the first embodiment in that an adaptor side facing surface 32 of an adaptor side electrode terminal 30 (only the anode side is shown in the diagram) of a pair of adaptor side electrode terminals, and an insertion portion side facing surface 47 of an insertion portion side electrode terminal 43 (only the anode side is shown in the diagram) of a pair of insertion portion side electrode terminals, that an endoscope 50 according to the present embodiment has, are arranged so as to face each other in a direction angled at a predetermined angle α with respect to a central axis C of an adaptor 51 that is a direction in which an insertion portion 52 is attached to and removed from the adaptor 51.

In a lens cylinder section 53 of the adaptor 51, a first plane surface 56a of a side surface section 56 of a depressed section 55 formed in a proximal end surface 53b of a base section 53A, is formed at an angle with the central axis C1, from a bottom section 20 of the depressed section 55 towards a proximal end section 53b of the base section 53A so that a substantially D shaped cross-sectional shape successively becomes greater. The adaptor side electrode terminal 30 and a cathode side terminal (not shown) are arranged side by side in a direction orthogonal to the central axis C1 on this first plane surface 56a.

On the other hand, in order to maintain the adaptor side facing surface 32 and the insertion portion side facing surface 47 in a state of facing each other, a second plane surface 60a of an insertion-portion-side side surface 60 of a protruding section 58 of a main body distal end section 57 is formed at an angle with respect to the central axis C2, which is parallel with the central axis C1, so that a substantially D shaped cross-sectional shape successively becomes smaller towards an distal end surface 61 of the protruding section 58.

Next, the procedures and effects when using the endoscope 50 according to the present embodiment is described.

First, as with the first embodiment, the protruding section 58 is inserted into the depressed section 51, with the engaging section 48B (see FIG. 2) of the depressed section 55 of the adaptor 51 engaged with the groove section 48A of the protruding section 58 of the main body distal end section 57.

At this time, immediately after insertion has begun, since the first plane surface 56a and the second plane surface 60a are angled at the same angle with respect to the central axis C, the adaptor side facing surface 32 of the adaptor side electrode terminal 30 has not yet come into contact with the insertion portion side facing surface 47 of the insertion portion side electrode terminal 43.

Then the male screw section 33 is screwed into the female screw section 22 of the connection ring 21 of the adaptor 51 to bring the distal end surface 61 of the protruding section 58 into contact with the bottom section 20 of the depressed section 55. At this time, since the distances from the central axis C to the adaptor side facing surface 32 and to the insertion portion side facing surface 47 are substantially equal, or, the distance to the insertion portion side facing surface 47 is slightly greater than the distance to the adaptor side facing surface 32, an area of their contact successively increases from the moment the adaptor side facing surface 32 and the insertion portion side facing surface 47 begin to come in contact with each other until screwing in has been completed.

As described above, the area of contact reaches a maximum when screwing is complete, and the adaptor side electrode terminal 30 and the insertion portion side electrode terminal 43 are respectively conducted and the adaptor 51 and the main body distal end section 57 are electrically connected.

After that, as with the first embodiment, the insertion portion 52 is inserted into a lumen (not shown), which is a subject to be observed, to cry out a predetermined observation.

When the observation is finished, the insertion portion 52 is withdrawn from the lumen, and the connection ring 21 and main body distal end section 57 are unscrewed. At this time, the adaptor side facing surface 32 of the adaptor side electrode terminal 30 and the insertion portion side facing surface 47 of the insertion portion side electrode terminal 43 separate, and the adaptor 51 is electrically disconnected from the insertion portion 52.

According to the endoscope 50, even if the facing surfaces 32 and 47 of the adaptor side electrode terminals 30 and the insertion portion side electrode terminals 43 are not brought into contact at the beginning of attachment of the insertion portion 52 to the adaptor 51, they can be brought into surface-contact when the adaptor 51 has been fully attached. Therefore, even if attachment and detachment of the adaptor 51 are repeatedly carried out, wear of the electrode terminals 30 and 43 can be suitably suppressed.

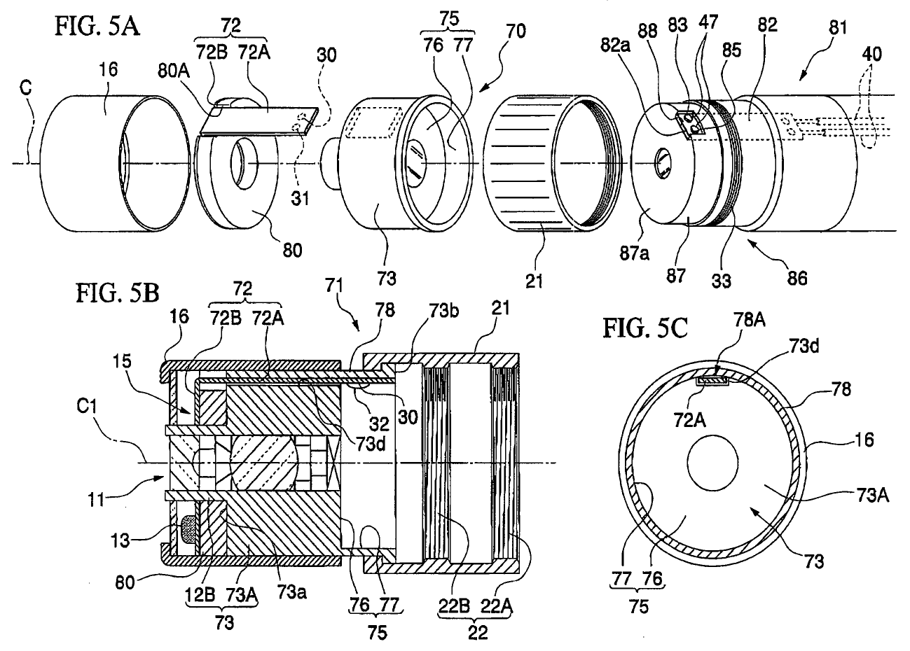

Next, a third embodiment is described, with reference to FIG. 5A to 5C.

Components similar to those in the other embodiments described above are given same reference symbols, and descriptions thereof are omitted.

The third embodiment differs from the first embodiment in that a flexible substrate 72 in an adaptor 71 of an endoscope 70 according to the present embodiment comprises an extension section 7A that is formed in a strip shape and extends from a distal end surface 73a side to a proximal end surface 73b along a side surface of a lens cylinder section 73, and the pair of adaptor side electrode terminals 30 and 31 are arranged on a wiring pattern (not shown) formed on the extension section 72A instead of on the pair of adaptor side terminal substrates 27 and 28.

A depressed section 75 formed in the base section 73A of the lens cylinder section 73 has a substantially circular shaped bottom section 76 having a diameter smaller than that of the base section 73A, and a portion between a side surface section 77 and the outer circumferential surface of the base section 73A forms a thin ring shaped wall section 78.

A through hole 73d arranged in the base section 73A is formed along the central axis C1 so as to pass through the bottom section 76 in proximity to the side surface section 77 to the distal end surface 73a of the lens cylinder section 73.

The through hole 73d is formed in a size that allows insertion of the extension section 72A.

An engaging section of an aligning section (not shown) is arranged on the side surface section 77 of the depressed section 75.

The flexible substrate 72 further comprises a disc section 72B on which a LED chip 13 is arranged, and the extension section 72A is formed so as to project from one portion of the disc section 72B and it is bent so as to lie flexibly along a side surface of an aluminum substrate 80. The pair of adaptor side electrode teals 30 and 31 is arranged in a line in a direction orthogonal to the central axis C1 on a distal end section of the extension section 72A, and they are electrically connected to the LED chip 13 arranged on the disc section 72B via a wiring pattern (not shown).

The extension section 72A has a substantially rectangular cross-sectional shape. As a result, since the side surface section 77 of the depressed section 75 of the lens cylinder section 73 is a curved surface and the side surface of the extension section 72A is a plane surface, a gap 78A is formed between the extension section 72A and the through hole 73d when the extension section 72A is inserted into the through hole 73d.

No hole such as the insert hole 25a mentioned in the first embodiment is formed on the aluminum substrate 80, instead, the outer diameter of the aluminum substrate 80 is formed to be substantially equal to that of the base section 73A of the lens cylinder section 73. A groove 80A having a shape that allows the extension section 72A of the flexible substrate 72 to be engaged and embedded in the aluminum substrate 80, is formed in the side space of the aluminum substrate 80.

A strip shaped insertion portion side flexible substrate 82 is connected to the electric wires 40 arranged on an insertion portion 81, and a pair of insertion portion side electrode terminals 83 and 85 are arranged in a line on a distal end section of the insertion portion side flexible substrate 82, in a direction orthogonal to the central axis C2 direction.

A cylinder section 87 arranged on a main body distal end section 86 of the insertion portion 81 has an outer diameter that can be fitted into the depressed section 75. A distal end surface 87a of the cylinder section 87 can be brought into contact with the bottom section 76 of the depressed section 75. A cutaway section 88 of sufficient size to engage the extension section 72A of the flexible substrate 72 and embed it into the cylinder section 87, is formed in the side surface of the cylinder section 87.

The cutaway section 88 is formed having a depth such that an end section 82a of the insertion portion side flexible substrate 82 extending within the cylinder section 87 can be mounted therein, and such that the distances from the central axis C to the adaptor side facing surface 32 and to the insertion portion side facing surface 47 are substantially equal, or, the distance to the insertion portion side facing surface 47 is slightly greater than the distance to the adaptor side facing surface 32.

Next, the procedures and effects when using the endoscope 70 according to the present embodiment is described.

First, with the main body distal end section 86 being engaged with the adaptor 71 by the aligning section (not shown), the cylinder section 87 of the main body distal end section 86 is inserted into the connection ring 21. At this time, as with the first embodiment, since the distances from the central axis C to the adaptor side facing surface 32 and to the insertion portion side facing surface 47 are substantially equal, or, the distance to the insertion portion side facing surface 47 is slightly greater than the distance to the adaptor side facing surface 32, the adaptor side facing surface 32 and the insertion portion side facing surface 47 begin to come into contact with each other. Then, the distal end surface 87a of the cylinder section 87 and the bottom section 76 of the depressed section 75 of the lens cylinder section 73 are brought into contact with each other by screwing the male screw section 33 and the female screw section 22 together.

Since the pair of adaptor side electrode terminals 30 and 31 are arranged on the extension section 72A of the flexible substrate 72 arranged along the side surface of the lens cylinder section 73, when attaching the adaptor 71 the extension section 72A elastically deforms outward in a radial direction within the gap 78A when the pair of insertion portion side electrode terminals 83 and 85 has come into contact with the pair of adaptor side electrode terminals 30 and 31.

Thus, the adaptor side facing surface 32 and the insertion portion side facing surface 47 come in contact with each other completely, with contact pressure between the facing surfaces 32 and 47 being adjusted by the elastic deformation of the extension section 72A.

After that, the operation is similar to that in the first embodiment.

According to the endoscope 70, effects similar to those achieved in the first embodiment can be achieved.

In particular, a frictional force that occurs between the facing surfaces 32 and 47 can be adjusted and the facing surfaces 32 and 47 can be brought into a more suitable contact condition.

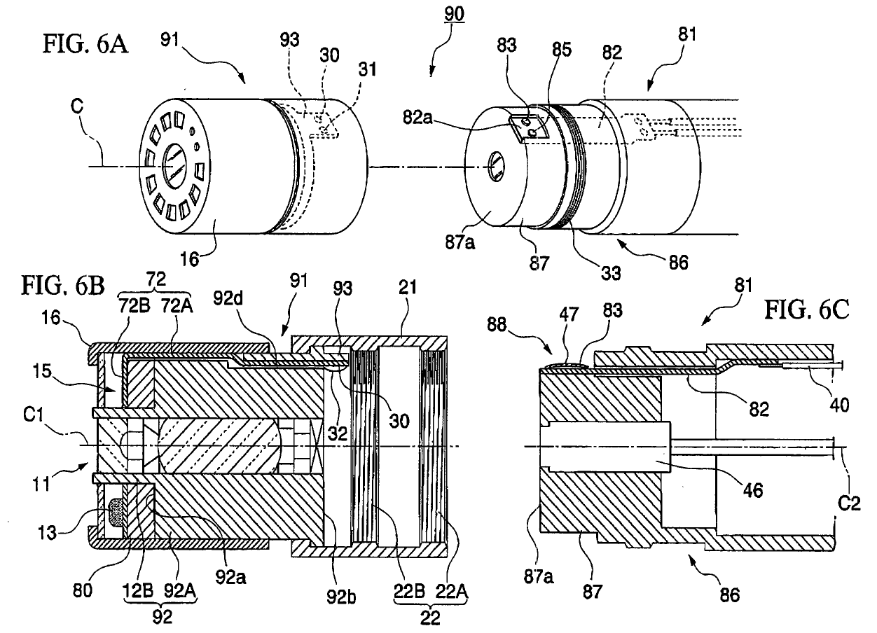

Next, a fourth embodiment is described, with reference to FIG. 6A to 6C.

Components similar to those in the other embodiments described above are given the same reference symbols, and descriptions thereof are omitted.

The fourth embodiment differs from the third embodiment in that whereas the depressed section 75 is provided on the base section 73A of the lens cylinder section 73 in the adaptor 71 of the endoscope 70 according to the third embodiment described above, on a base section 92A of a lens cylinder section 92 in an adaptor 91 of an endoscope 90 according to the present embodiment, a support section 93 is arranged, having a plate width substantially equal to that of the extension section 72A of the flexible substrate 72, and being formed to project from a proximal end surface 92b of a lens cylinder section 92 and being bendable in a radial direction of the lens cylinder section 92.

The support section 93 has a thickness that allows a gap between itself and an inner wall surface of the connection ring 21, and is formed in a shape that allows it to be embedded in the cutaway section 88 arranged on the cylinder section 87 of the main body distal end section 86.

A through hole 92d of the lens cylinder section 92 is formed not from a distal end surface 92a of the base section 92A but from one portion of the side surface towards the proximal end surface 92b in the direction of the central axis C1 within Me base section 92A.

When inserted into the through hole 92d, the extension section 72A of the flexible substrate 72 projects from the proximal end surface 92b and is supported by an inner surface of the support section 93.

The engaging section of the aligning section (not shown) is arranged on an inner surface of the outside member 16, and the groove section (not shown) is provided on the cylinder section 87 of the main body distal end section 86.

The procedures and effects when using the endoscope 90 is described.

As with the third embodiment, the adaptor side facing surface 32 and the insertion portion side facing surface 47 begin to come into contact with each other. The the distal end surface 87a of the cylinder section 87 and the proximal end surface 92b of the base section 92A of the lens cylinder section 92 are brought in contact with each other by screwing the male screw section 33 and the female screw section 22 together.

At this time, the support section 93 bends outward in a radial direction within the gap when the pair of insertion portion side electrode terminals 83 and 85 and the pair of adaptor side electrode terminals 30 and 31 have come in contact with each other on insertion of the adaptor 91.

Thus, the adaptor side facing surface 32 and the insertion portion side facing surface 47 come in contact with each other completely, with contact pressure between the facing surfaces 32 and 47 being adjusted by the bending of the support section 93.

After that, the operation is similar to that in the first embodiment.

According to the endoscope 90, since the support section 93 is shorter than the wall section 78 in the crosswise direction with respect to the central axis C, and is easier to bend compared to the endoscope 70 according to the third embodiment, the extension section 72A of the flexible substrate 72 can be deformed more suitably, and frictional force that occurs when the adaptor side facing surface 32 and the insertion portion side facing surface 47 are in contact, can be easily adjusted.

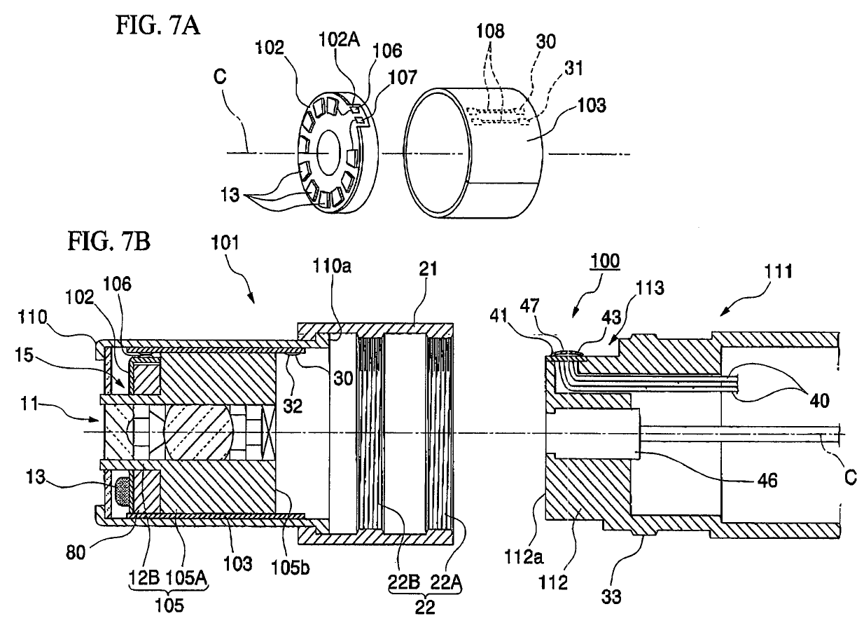

Next, a fifth embodiment is described, with reference to FIGS. 7A and 7B.

Components similar to those in the other embodiments described above are given the same reference symbols, and descriptions thereof are omitted.

The fifth embodiment differs from the third and fourth embodiments in that a cylindrical shaped flexible substrate 103 to be electrically connected to a flexible substrate 102, is arranged on an adaptor 101 of an endoscope 100.

A proximal end surface 105b of a base section 105A of a lens cylinder section 105 is a flat surface.

The length of an extension section 102A of the flexible substrate 102 is substantially equal to the thickness of an aluminum substrate 80 in the direction of the central axis C1, and a pair of connection terminals 106 and 107 for LED chips 13, is formed on an outer surface thereof.

The cylindrical shaped flexible substrate 103 is formed from a sheet substrate wrapped around in a cylindrical shape so as to cover the outer circumferential surfaces of the extension section 102A and the lens cylinder section 105. The length of the cylindrical shape flexible substrate 103 in the direction of the central axis C1 is formed to be longer than the combined length of the aluminum substrate 80 and the lens cylinder section 105 when these are joined in the central axis C1 direction. A pattern 108 that extends in the central axis C1 direction and whose one end side contacts with a pair of connection terminals 106 and 107, is arranged on an inner surface of the cylindrical shape flexible substrate 103.

The pair of adaptor side electrode terminals 30 and 31 is arranged at the other end side of the wiring pattern 108.

A flange section 110a with which the connection ring 21 engages, is arranged on a proximal end of an outside conductor 110.

In a main body distal end section 111, as with the case of the endoscope 1 according to the first embodiment, electric wires 40 are arranged to extend to the proximity of a distal end of a cylinder section 112. On the side surface of the cylinder section 112 is arranged a cutaway section 113 in which the insertion portion side terminal substrate 41 on which the insertion portion side electrode terminal 43 is arranged, and a cathode side insertion portion side teal substrate (not shown) on which a cathode side insertion portion side electrode terminal (not shown) is arranged, can both be mounted along the circumferential direction of the side surface of the cylinder section 112.

The procedures and effects when using the endoscope 100 is described.

As with the first embodiment, the adaptor side facing surface 32 and the insertion portion side facing surface 47 begin to come into contact with each other. Then, the distal end surface 112a of the cylinder section 112 and the proximal end surface 105b of the base section 105A of the lens cylinder section 105 are brought in contact with each other by screwing the male screw section 33 and the female screw section 22 together.

At this time, the insertion portion side electrode terminal 43 and the adaptor side electrode terminal 30 begin to come into contact with each other in the direction of the central axis C, and the adaptor side facing surface 32 and the insertion portion side facing surface 47 come into contact with each other completely.

Thus, the adaptor 101 is electrically connected to the main body distal end section 111.

According to the endoscope 100, since the operation of the cylindrical shape flexible substrate 103 according to the present embodiment is similar to that of the wall section 78 of the lens cylinder section 105 according to the third embodiment, an operation and effect similar to that of the endoscope 70 according to the third embodiment can be achieved.

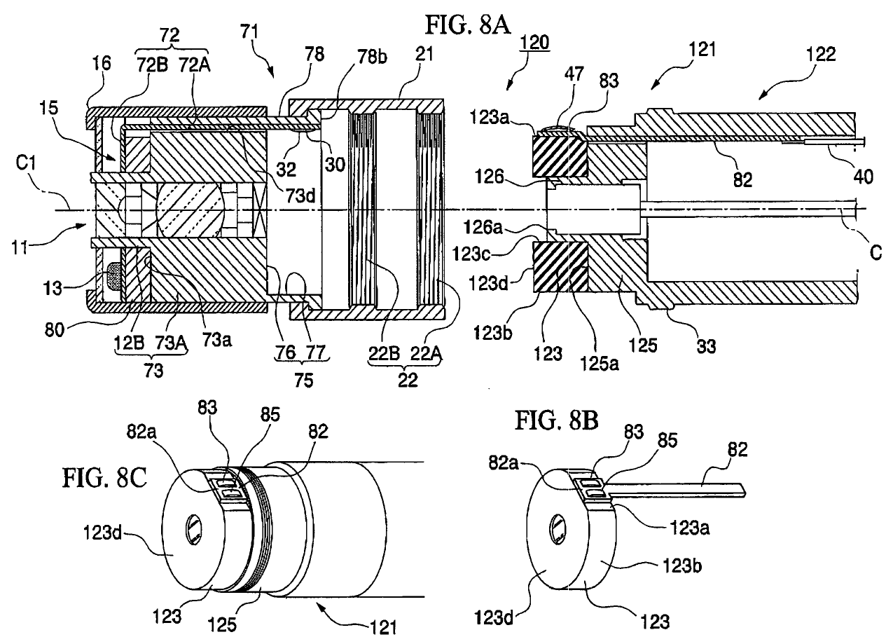

Next, a sixth embodiment is described, with reference to FIG. 8A to 8C.

Components similar to those in the other embodiments described above are given the same reference symbols, and descriptions thereof are omitted.

The sixth embodiment differs from the third embodiment in that a rubber member (biasing device) 123 is arranged on a main body distal end section 121 of an endoscope 120 according to the present embodiment, fitted to a cylinder wall 126 formed to project from a distal end surface 125a of a cylinder section 125, to successively increase a pressing pressure between the adaptor side electrode terminal (only the anode side is shown in the diagram) 30 of the pair of adaptor side electrode terminals, and the insertion portion side electrode terminal 83 of the pair of insertion portion side electrode terminals 83 and 85 when the adaptor 71 is attached to an insertion portion 122.

The rubber member 123 is formed in a sectional D shape having a first side surface 123a formed from a flat surface and a second side surface 123b formed from an arc surface having a diameter substantially equal to that of the cylinder section 125, and a hole 123c to which the cylinder wall 126 of the cylinder section 125 is fitted is arranged in a central section of the rubber member 123.

A distal end surface 123d of the rubber member 123 is arranged so as to project slightly from the distal end of the cylinder section 125 of the main body distal end section 121 when the rubber member 123 is fitted to the cylinder wall 126.

An end section 82a of an insertion portion side flexible substrate 82 is mounted on the fist side surface 123a of the rubber member 123. At this time, the distance from the central axis C to an insertion portion side facing surface 47 is substantially equal to the distance from the central axis C to an adaptor side facing surface 32.

Next, the procedures and effects when using the endoscope 120 according to the present embodiment is described.

First, the cylinder section 125 of the main body distal end section 121 is inserted into the connection ring 21 and the outside member 16 of the adaptor 71 until the distal end surface 123d of the rubber member 123 has come in contact with the bottom section 76 of the depressed section 75 of the lens cylinder section 73.

At this time, the insertion portion side facing surface 47 and the adaptor side facing surface 32 are opposed to each other, however, they are not yet in contact with each other.

Then, the male screw section 33 of the main body distal end section 121 is screwed together with the female screw section 22 of the connection ring 21.

At this time, a compressive force in the central axis C direction is loaded onto the rubber member 123, and the rubber member 123 is elastically deformed outward in the radial direction by a resistance force from the cylinder section 125, so that the outer diameter of the rubber member 123 increases. As a result, the distance from the central axis C to the insertion portion side facing surface 47 changes as the insertion portion side facing surface 47 moves outward in the radial direction. Then, the insertion portion side facing surface 47 begins to come into contact with the adaptor side facing surface 32.

In this way, the area of contact successively increases by the time screwing has been completed and the area of contact reaches a maximum when screwing has been completed, and the adaptor 71 and the insertion portion 122 are electrically connected.

According to the endoscope 120, the area of contact between the adaptor side electrode terminal 30 and the insertion portion side electrode terminal 83 can be increased by deforming the rubber member 123 outward in the radial direction by pressing the rubber member 123, and each of the corresponding facing surfaces 32 and 47 can be reliably brought into contact with each other. At this time, since the insertion portion side facing surface 47 does not come in contact with the adaptor side facing surface 32 unless the rubber member 123 is compressed, wear in the pair of adaptor side electrode terminals and the pair of insertion portion side electrode terminals can be suitably suppressed.

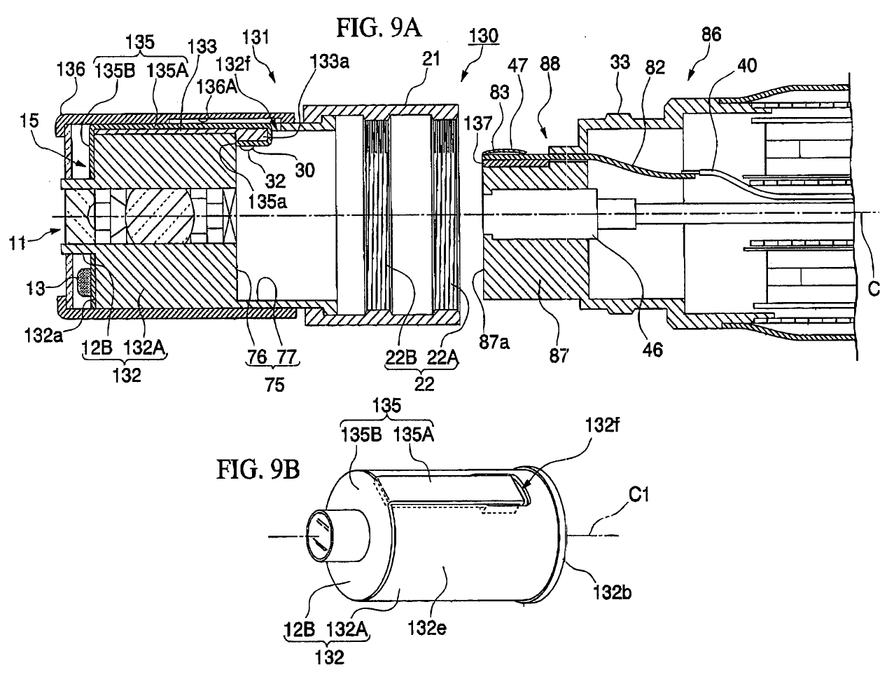

Next, a seventh embodiment is described, with reference to FIG. 9A and 9B.

Components similar to those in the other embodiments described above are given the same reference symbols, and descriptions thereof are omitted.

The seventh embodiment differs from the third embodiment in that a plate spring section (biasing device, spring member) 133 that is bent in the radial direction to give a pressing force, is arranged in a lens cylinder section 132 in an adaptor 131 of an endoscope 130 according to the present embodiment.

The plate spring section 133 is formed by cutting out one portion of a side surface 132e along the central axis C1 from a distal end surface 132a of a base section 132A of the lens cylinder section 132 to a proximal end surface 132b. Meanwhile, a hole section 132f is formed in the side surface 132e of the lens cylinder section 132 where the plate spring section 133 has been cut out. The plate spring section 133 can bend in the radial direction within the hole section 132f.

An end section 133a of the plate spring section 133 is formed so as to project into the depressed section 75.

An extension section 135a of the flexible substrate 135 is arranged along the outer circumferential surface of the plate spring section 133 and the extension section 135a is fixed by being folded back at the end section 133a of the plate spring section 133.

The adaptor side electrode terminal 30 (only the anode side is shown in the diagram) is arranged on a top surface of the extension section 135a folded back into the depressed section 75.

A disc section 135B of the flexible substrate 135 is arranged on the distal end surface 132a of the base section 132A of the lens cylinder section 132 and not on an aluminum substrate.

On an inner surface of an outside member 136, a depressed section 136A is formed to ensure that the range regulated by the outside member 136 allows at least a bending movement in the case where the plate spring section 133 bends and deforms.

A sheet elastic body 137 is arranged between the insertion portion side flexible substrate 82 and the cutaway section 88 in the main body distal end section 86. The thickness of this elastic body 137 is adjusted so that the distance from the central axis C to the adaptor side facing surface 32 becomes shorter than the distance from the central axis C to the insertion portion side facing surface 47, to a degree that the adaptor 131 can be attached to the main body distal end section 86.

Next, the procedures and effects when using the endoscope 130 according to the present embodiment is described.

First, the cylinder section 87 of the main body distal end section 86 is inserted into the depressed section 75 of the adaptor 131 along the aligning section (not shown).

At this time, the adaptor side facing surface 32 of the adaptor side electrode terminal 30 begins to come into contact with the insertion portion side facing surface 47 of the insertion portion side electrode terminal 83. At this time, since a radial direction outward force is loaded onto the adaptor side electrode terminal 30 from the insertion portion side electrode terminal 83, the plate spring section 133 bends outward in the radial direction within the hole section 132f, with the adaptor side facing surface 32 and the insertion portion side facing surface 47 in contact with each other.

Meanwhile, since the inward force in the radial direction is loaded onto the inset section side electrode terminal 83 from the adaptor side electrode terminal 30, the insertion portion side flexible substrate 82 bends inward in the radial direction, compressing the elastic body 137.

The male screw section 33 is screwed into the female screw section 22 of the connection ring 21 of the adaptor 131 to bring the distal end space 87a of the cylinder section 87 into contact with the bottom section 76 of the depressed section 75 of the lens cylinder section 132. At this time, the end section 133a of the bent plate spring section 133 comes in contact with the depressed section 136A of the outside member 136. Then, a radial direction inward force acts on it from the outside member 136.

Therefore, a constant force is maintained, and the area of contact between the adaptor side facing surface 32 and the insertion portion side facing surface 47 is maintained constant, and the pair of adaptor side electrode terminals and the pair of insertion portion side electrode terminals are respectively conducted and the adaptor 131 and the main body distal end section 86 are electrically connected.

According to the endoscope 130, the area of contact between the pair of adaptor side electrode terminals and the pair of insertion portion side electrode terminals can be successively increased while adjusting the force by deforming the plate spring section 133 when attaching the adaptor, and wear of the pair of adaptor side electrode terminals and the pair of insertion portion side electrode terminals can be suitably suppressed.

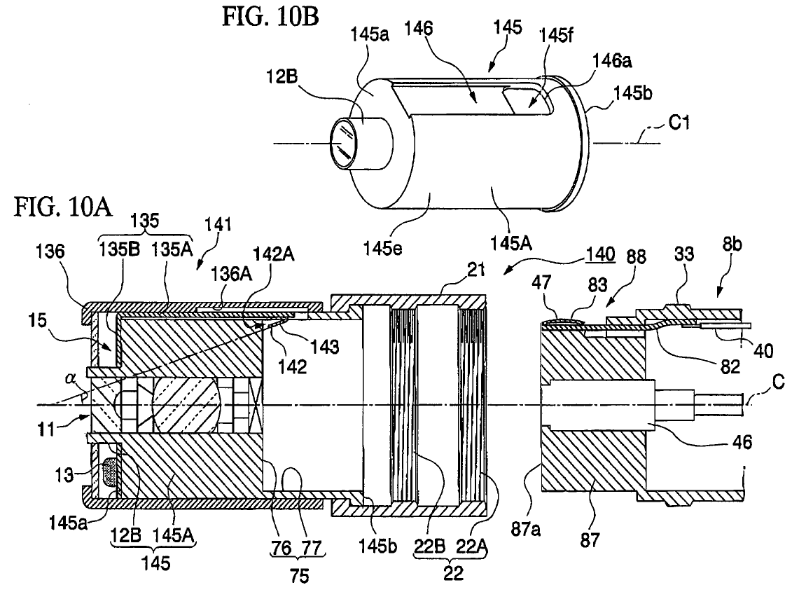

Next, an eighth embodiment is described, with reference to FIGS. 10A and 10B.

Components similar to those in the other embodiments described above are given the same reference symbols, and descriptions thereof are omitted.

The eighth embodiment differs from the seventh embodiment in that adaptor side electrode terminals 142 (only the anode side is shown in the diagram) of a pair of adaptor side electrode terminals in an adaptor 141 of an endoscope 140 according to the present embodiment, are formed as plate spring elastic members, and an adaptor side facing surface 143 is arranged at a constant angle α with respect to the central axis C.

The adaptor side electrode terminal 142 is formed connected to the extension section 135A of the flexible substrate 135 and is bent back in the inward radial direction to a constant angle α with respect to the extension section 135A of the flexible substrate 135. A space section 142A is formed at the folding back portion.

An engaging groove 146 having a predetermined length along the central axis C1 from a distal end surface 145a towards a proximal end surface 145b is arranged in one portion of a side surface 145e of a base section 145A of a lens cylinder section 145. This engaging groove 146 has a cross-sectional shape into which the extension section 135A of the flexible substrate 135 can be fitted and embedded. A hole section 145f through which the adaptor side electrode terminal 142 arranged on the end section of the extension section 135A can pass from the outer surface of the lens cylinder section 145 to the side surface section 77, is formed in an end section 146a of the engaging groove 146.

The adaptor side facing surface 143 of the adaptor side electrode terminal 142 and the inset section side facing surface 47 of the insertion portion side electrode terminal 83 do not oppose each other until the adaptor 141 has been attached.

Next, the procedures and effects when using the endoscope 140 according to the present embodiment is described.

First, the cylinder section 87 of the main body distal end section 86 is inserted into the depressed section 75 of the adaptor 141 along the aligning section (not shown).

At this time, the adaptor side facing surface 143 and the insertion portion side facing surface 47 begin to come in contact with each other. At this time, a radial direction outward force is loaded onto the adaptor side electrode terminal 142, and at first, the extension section 135A of the flexible substrate 135 bends and deforms outward in the radial direction.

As attachment proceeds further, the extension section 135A of the flexible substrate 135 comes into contact with the depressed section 136A of the outside member 136 and stops deforming. Thereafter, the adaptor side electrode terminal 142 elastically deforms in a direction in which the space section 142A becomes smaller. The area of contact between the adaptor side facing surface 143 and the insertion portion side facing surface 47 of the insertion portion side electrode terminal 83 successively increases, and the adaptor side electrode terminal 142 and the insertion portion side electrode terminal 83 become substantially parallel with each other.

The male screw section 33 is screwed into the female screw section 22 of the connection ring 21 of the adaptor 141 to bring the distal end surface 87a of the cylinder section 87 into contact with the bottom section 76 of the depressed section 75 of the lens cylinder section 145. At this time, the adaptor side facing surface 143 and the insertion portion side facing surface 47 oppose to and come in contact with each other with the area of contact between them maintained constant, and the pair of adaptor side electrode terminals and the pair of insertion portion side electrode terminals are respectively conducted and the adaptor 141 and the main body distal end section 86 are electrically connected.

According to the endoscope 140, by attaching the adaptor 141 to the main body distal end section 86 and deforming the pair of the plate spring adaptor side electrode terminals, the pair of adaptor side electrode terminals and the pair of insertion portion side electrode terminals can be brought in contact with each other while increasing the area of contact between them, and wear of both of them can be suitably suppressed.

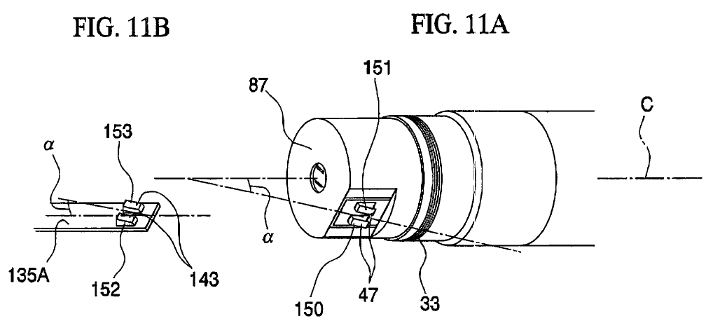

As shown in FIG. 11A, a pair of insertion portion side electrode terminals 150 and 151 may be formed in a form of a plate spring that is angled at a constant angle α with respect to the central axis C. At this time, as shown in FIG. 11B, adaptor side electrode terminals 152 and 153 may be formed as metal bodies with the adaptor side facing surfaces 143 opposing to the insertion portion side facing surfaces 47 at the constant angle α with respect to the central axis C.

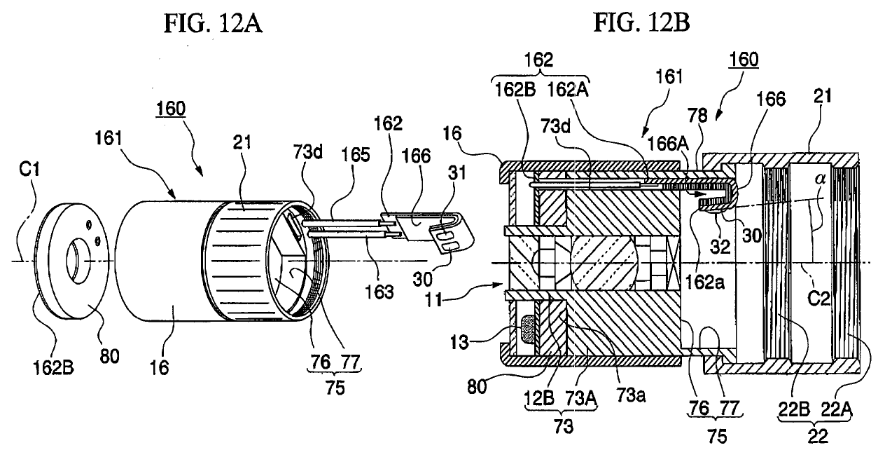

Next, a ninth embodiment is described, with reference to FIGS. 12A and 12B.

Components similar to those in the other embodiments described above are given the same reference symbols, and descriptions thereof are omitted.

The ninth embodiment differs from the eighth embodiment in that in an adaptor 161 of an endoscope 160 according to the embodiment, an extension section 162A and a disc section 162B of a flexible substrate 162, are separated from each other and they are both connected by a pair of electric wires 163 and 165.

A plate spring section 166 folded back inward in the radial direction at a constant angle α with respect to the central axis C1, is connected to a proximal end of the extension section 162A. A space section 166A is formed in a portion where the plate spring section 166 is folded back. An end section side of the extension section 162A of the flexible substrate 162 is folded back along the plate spring section 166.

The pair of adaptor side electrode terminals 30 and 31 is arranged on a surface of an end section 162a of the extension section 162A that has been folded back to face inward in the radial direction.

According to the endoscope 160, since the pair of adaptor side electrode terminals 30 and 31 are arranged on a top surface of the plate spring section 166 connected to the flexible substrate 162, and the extension section 162A and the disc section 162B are connected to the pair of the electric wires 163 and 165, an operation and effect similar to that of the eighth embodiment can be achieved.

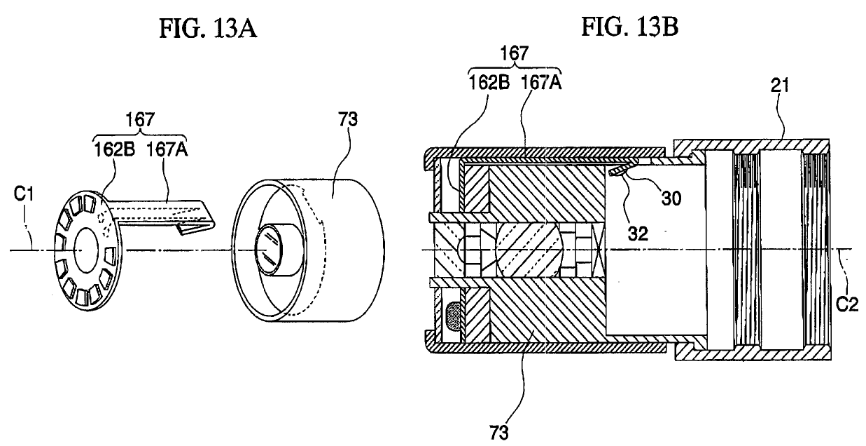

Furthermore, instead of connecting the flexible substrate 162 and the plate spring section 166 with the pair of the electric wires 163 and 165, as shown in FIGS. 13A and 13B, the proximal end side of the extension section 162A may be folded back inward in the radial direction and a flexible substrate 167 having a plate spring extension section 167A may be provided, while the disc section 162B of the flexible substrate 162 is left as it is.

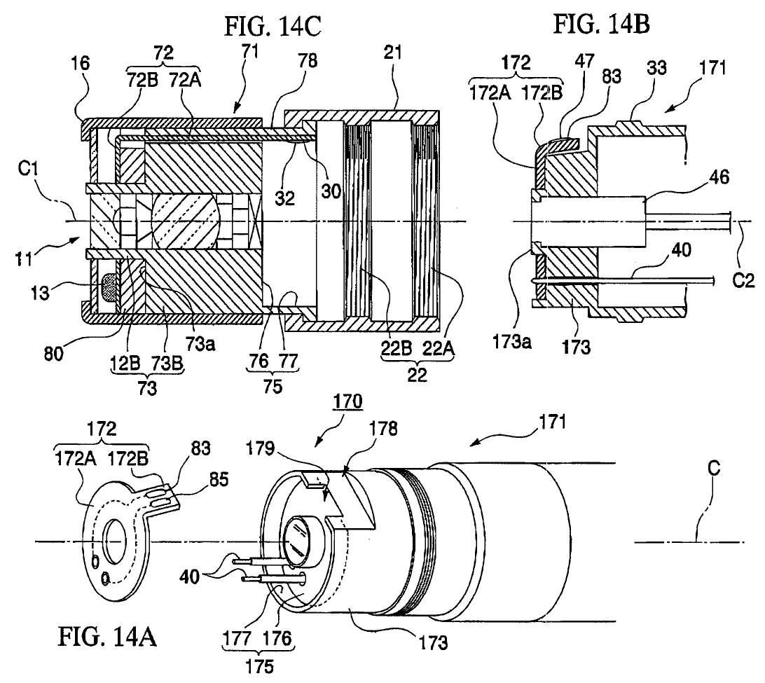

Next, a tenth embodiment is described, with reference to FIG. 14A to 14C.

Components similar to those in the other embodiments described above are given the same reference symbols, and descriptions thereof are omitted.

The tenth embodiment differs from the the embodiment in that an insertion portion side flexible substrate 172 of a main body distal end section 171 of an endoscope 170 according to the present embodiment is arranged on a distal end of a cylinder section 173.

The insertion portion side flexible substrate 172 comprises a disc section 172A formed in a disc shape through which electric wires 40 that extend through the cylinder section 173 can pass, and a terminal section 172B that extends from one portion of the periphery of the disc section 172A at a constant angle with respect to the central axis C2. The disc section 172A and the terminal section 172B are integrally formed, and the terminal section 172B can bend like a plate spring with respect to the disc section 172A. The pair of insertion portion side electrode terminals 83 and 85 are arranged on a top surface of the terminal section 172B.

An insertion portion side depressed section 175 that can accommodate the insertion portion side flexible substrate 172 is formed in a distal end surface 173a of the cylinder section 173 of the main body distal end section 171. The insertion portion side depressed section 175 comprises a bottom section 176 and a side surface section 177 as is the case with the depressed section 75 of the adaptor 71, and a second cutaway section 179 for directly connecting the bottom section 176 and the cutaway section 178 is formed in one portion of the side surface section 177. Furthermore, a groove section of an aligning section (not shown) is formed in the side surface section 177 of tie insertion portion side depressed section 175.

Next, the procedures and effects when using the endoscope 170 according to the present embodiment is described.

First, the cylinder section 173 of the main body distal end section 171 is inserted into the depressed section 75 of the adaptor 71 along the aligning section (not shown).

At this time, the insertion portion side facing surface 47 and the adaptor side facing surface 32 of the adaptor side electrode terminal 30 (only the anode side is shown in the diagram) begin to come in contact with each other. At this time, since a radial direction inward force is loaded onto the insertion portion side electrode terminal 83, the pair of insertion portion side electrode terminals 83 and 85 are pressed inward in the radial direction. Therefore, the terminal section 172B of the insertion portion side flexible substrate 172 bends in a direction along the central axis C, and an area of contact between the insertion portion side facing surface 47 and the adaptor side facing surface 32 increases as they become substantially parallel.

The male screw section 33 is screwed into the female screw section 22 of the connection ring 21 of the adaptor 71 to bring the distal end surface 173a of the cylinder section 173 into contact with the bottom section 76 of the depressed section 75 of the lens cylinder section 73. At this time, the adaptor side facing surface 32 and the insertion portion side facing surface 47 oppose to and come in contact with each other with the area of contact between them maintained constant, and the pair of adaptor side electrode terminals and the pair of insertion portion side electrode terminals are respectively conducted, and the adaptor 71 and the main body distal end section 171 are electrically connected.

According to the endoscope 170, since the pair of insertion portion side electrode terminals 83 and 85 are arranged on the terminal section 172B of the insertion portion side flexible substrate 172 that deforms like a plate spring with respect to the disc section 172A, by attaching the adaptor 71 to the main body distal end section 171 and deforming the terminal section 172B, the pair of adaptor side electrode terminals and the pair of insertion portion side electrode terminals can be brought into contact with each other while gradually increasing an area of contact between them, and wear of them can be suitably suppressed.

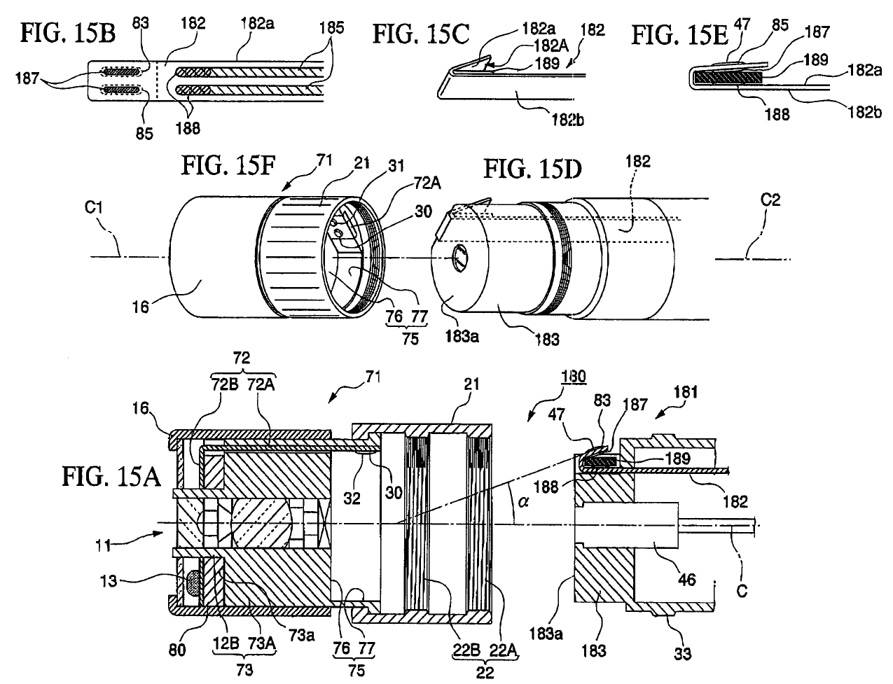

Next, an eleventh embodiment is described, with reference to FIG. 15A to 15F.

Components similar to those in the other embodiments described above are given the same reference symbols, and descriptions thereof are omitted.

The eleventh embodiment differs from the third embodiment in that an insertion portion side flexible substrate 182 of a main body distal end section 181 of an endoscope 180 according to the present embodiment is arranged so as to be folded back at a distal end of a cylinder section 183, as shown in FIG. 15A.

As shown in FIGS. 15B and 15C, the insertion portion side flexible substrate 182 is formed in a rectangular plane plate shape and comprises a first side surface 182a that is arranged to be in contact with the cylinder section 183 and upon which a wiring pattern 185 to be connected to the electric wires 40 is formed so as to extend to its distal end side; and a second side surface 182b that is a side surface opposite to the first side surface 182a and is arranged facing the outward radial direction of the cylinder section 183.

As shown in FIG. 15D, the distal end side of the insertion portion side flexible substrate 182 is folded back towards the first side surface 182a side in a direction angled at a constant angle α with respect to the central axis C2, and creates a space section 182A, and can elastically deform until the first side surface 182a comes into contact with itself.

The pair of insertion portion side electrode terminals 83 and 85 is arranged on the second side surface 182b so as to be separated from the distal end of the wiring pattern 185 by a predetermined distance as shown in FIG. 15E. First contact points 187 that correspond to the respective electrode terminals 83 and 85 are arranged in positions on the first side surface 182a where the pair of insertion portion side electrode terminals 83 and 85 are arranged.

Second contact points 188 that face the first contact points 187 when the distal end side of the insertion portion side flexible substrate 182 is folded back, are arranged on the distal end of the wiring pattern 185.

A conductive rubber sheet (biasing device, rubber member) 189 that has a conducting property in a pressed direction when pressed, is arranged in the space section 182A formed when the distal end section of the insertion portion side flexible substrate 182 is folded back, as shown in FIG. 15E.

The conductive rubber 189 here is constructed with conductive members such as nickel particles or gold plated metal particles or the like embedded in a dotted pattern in an insulating rubber material such as silicone rubber, and it is generally referred to as a dot type anisotropic conductive rubber and so forth. In this conductive rubber 189, when the rubber material, which is an elastic body, is pressed in a thickness direction, a conducting property between the conductive members increases as they become highly dense due to compressive deformation of the rubber material and thereby conduction in the thickness direction is allowed. At this time, since the rubber material is an insulating member, insulation in directions other than the thickness direction of the rubber material (for example, the circumferential direction) is still maintained.

Next, the procedures and effects when using the endoscope 180 according to the present embodiment is described.

First, the cylinder section 183 of the main body distal end section 181 is inserted into the depressed section 75 of the adaptor 71 shown in FIG. 15F along the aligning section (not shown).

At this time, the insertion portion side facing surfaces 47 of the pair of insertion portion side electrode terminals 83 and 85 and the adaptor side facing surfaces 32 of the pair of adaptor side electrode terminals 30 and 31 begin to come in contact with each other.

At this time, since a radial direction inward force is loaded onto the folded-back distal end portion of the insertion portion side flexible substrate 182, the distal end portion deforms in a direction which becomes substantially parallel with the central axis C2, making the space section 182A smaller. Then, the conductive rubber 189 is sandwiched by the first contact points 187 and the second contact points 188, and compressed and deformed.

As a result, a degree of contact between the conductive rubber 189 and the wiring pattern 185, and contact between the conductive rubber 189 and the pair of insertion portion side electrode terminals 83 and 85 increases, and the conducting property of the conductive rubber 189 in the radial direction increases to electrically connect the pair of insertion portion side electrode terminals 83 and 85 and the wiring pattern 185.

At the same time, the area of contact between the insertion portion side facing surface 47 and the adaptor side facing surface 32 successively increases.

The male screw section 33 is screwed into the female screw section 22 of the connection ring 21 of the adaptor 71 to bring the distal end surface 183a of the cylinder section 183 into contact with the bottom section 76 of the depressed section 75 of the lens cylinder section 73. At this time, the adaptor side facing surface 32 and the insertion portion side facing surface 47 oppose to and come in contact with each other with the area of contact between them maintained constant, and the pair of adaptor side electrode terminals 30 and 31 and the pair of insertion portion side electrode terminals 83 and 85 are conducted, respectively, and the adaptor 71 and the main body distal end section 181 are electrically connected.

According to the endoscope 180, when the pair of insertion portion side electrode terminals 83 and 85 are electrically connected with the wiring pattern 185 of the same insertion portion side flexible substrate 182, by pressing to deform the conductive rubber 189 in response to the attachment of the adaptor, the condition of electrical connection between the pair of insertion portion side electrode terminals 83 and 85 and the wiring pattern 185 can be gradually improved.

Meanwhile, since the conductive rubber 189 elastically deforms while the adaptor 71 is being attached to the main body distal end section 181, then as with the seventh embodiment, the pair of adaptor side electrode terminals 30 and 31 and the pair of insertion portion side electrode terminals 83 and 85 can gradually be brought in contact with each other while the area of their contact gradually increases and wear of both of them can be suitably suppressed.

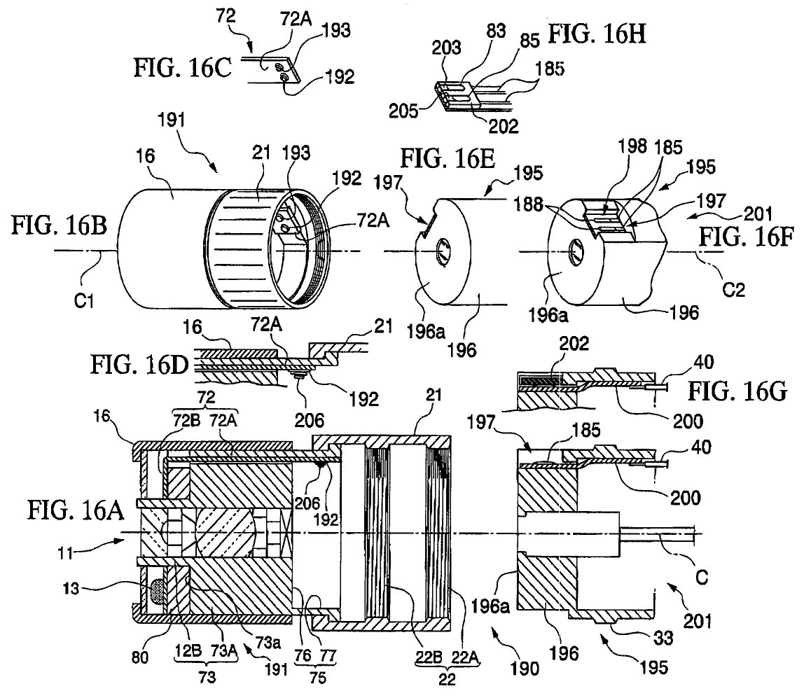

Next, a twelfth embodiment is described, with reference to FIG. 16A to 16G.

Components similar to those in the other embodiments described above are given the same reference symbols, and descriptions thereof are omitted.

The twelfth embodiment differs from the eleventh embodiment in that a pair of adaptor side electrode terminals 192 and 193 of an adaptor 191 of an endoscope 190 according to the present embodiment shown in FIGS. 16A and 16B are formed as coil springs (elastic members), the diameters of which become smaller towards the inside in the radial direction, and are arranged on the extension section 72A of the flexible substrate 72 as shown in FIGS. 16C and 16D.

A cutaway depressed section 198 is formed inward in the radial direction on a top surface of a cutaway section 197 formed in a cylinder section 196 of a main body distal end section 195 as shown in FIGS. 16E and 16F.

A distal end of an insertion portion side flexible substrate 200 is arranged so as to project from a proximal end side of an insertion portion 201 into the cutaway depressed section 198.

As shown in FIG. 16G, a conductive rubber 202 is mounted in the cutaway depressed section 198. A pair of metal plates 203 and 205 are arranged on this conductive rubber 202 as shown in FIG. 16H, with the middle portion of the plates folded back at the distal end of the conductive rubber 202, one end of the inward radial direction side of the plates being in contact with the wiring pattern 185 of the insertion portion side flexible substrate 200, and the other end of the outward radial direction side of the plates having the pair of insertion portion side electrode terminals 83 and 85 arranged thereon. In other words, the pair of insertion portion side electrode terminals 83 and 85 and the insertion portion side flexible substrate 200 can be electrically brought in contact with each other via the pair of the metal plates 203 and 205 and the conductive rubber 202.

The thickness of the conductive rubber 202 when it is not compressed is adjusted so that the distance between the pair of insertion portion side electrode terminals 83 and 85 and the central axis C2 is greater than the distance between the central axis C1 and the distal end section 206 of the adaptor side electrode terminals 192 and 193, and shorter than the distance from the central axis C1 to an attachment surface of the pair of adaptor side electrode terminals 192 and 193.

Next, the procedures and effects when using the endoscope 190 according to the present embodiment is described.

First, the cylinder section 196 of the main body distal end section 195 is inserted into the deposed section 75 of the adaptor 191 along the aligning section (not shown), and the pair of insertion portion side electrode terminals 83 and 85 and the pair of adaptor side electrode terminals 192 and 193 begin to come into contact with each other.

A radial direction inward force from the pair of insertion portion side electrode terminals 83 and 85 is loaded onto the pair of adaptor side electrode terminals 192 and 193, causing them to compress and deform. At this time, since the coil diameters of the pair of adaptor side electrode terminals 192 and 193 become successively smaller, the area of their contact with the pair of insertion portion side electrode terminals 83 and 85 successively increases as they are compressed.

Meanwhile, the pair of insertion portion side electrode terminals 83 and 85 receives a radial direction inward pressing force from the pair of adaptor side electrode terminals 192 and 193. As a result, the conductive rubber 202 is compressed in the plate thickness direction via the pair of the metal plates 203 and 205, and the pair of the metal plates 203 and 205 is pressed to the insertion portion side flexible substrate 200 side. Thus, while the area of their contact successively increases, the conducting property of the conductive rubber 202 in the compression direction increases.

The male screw section 33 is screwed into the female screw section 22 of the connection ring 21 of the adaptor 191 to bring the distal end surface 196a of the cylinder section 196 into contact with the bottom section 76 of the depressed section 75 of the lens cylinder section 73. At this time, the area of contact between the pair of adaptor side electrode terminals 192 and 193 and the pair of insertion portion side electrode terminals 83 and 85 reaches a maximum, and the adaptor 191 and the main body distal end section 195 are electrically connected.

According to the endoscope 190, as with the eleventh embodiment, the pair of insertion portion side electrode terminals 83 and 85 comes into electrical contact with the wiring pattern 185 of the insertion portion side flexible substrate 200 via the conductive rubber 202, and their contact condition can be gradually improved. At this time, since the pair of adaptor side electrode terminals 192 and 193 itself elastically deforms, the area of contact between the pair of adaptor side electrode terminals 192 and 193 and the pair of insertion portion side electrode terminals 83 and 85 can be more smoothly increased while they are brought in contact with each other.

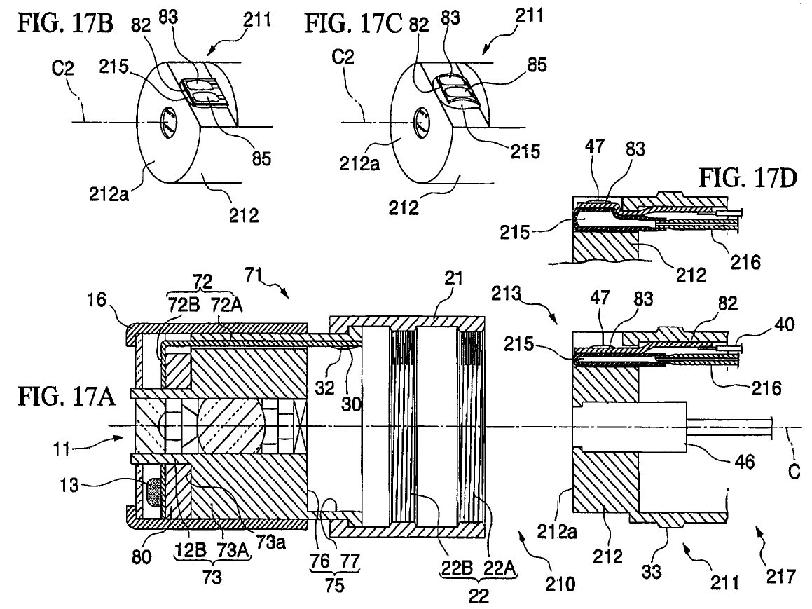

Next, a thirteenth embodiment is described, with reference to FIG. 17A to 17D.

Components similar to those in the other embodiments described above are given the same reference symbols, and descriptions thereof are omitted.

The thirteenth embodiment differs from the third embodiment in that a balloon (biasing device) 215 that bends the distal end side of the insertion portion side flexible substrate 82 exposed in a cutaway section 213 in the radial direction when it is supplied with air/air is removed to inflates it/deflate it, is arranged in the cutaway section 213 arranged in a cylinder section 212 of a main body distal end section 211 of an endoscope 210 according to the present embodiment as shown in FIG. 17A.

The distal end section of the insertion portion side flexible substrate 82 is mounted on the balloon 215. An air tube 216 for supplying air to or removing supplied air from the balloon 215, is arranged along the electric wires 40 within an insertion portion 217, one end of the air tube being connected to the balloon 215 and the other end connected to an air source (not shown).

The procedures and effects when using The endoscope 210 is described.

First, the cylinder section 212 of the main body distal end section 211 is inserted into the depressed section 75 of the adaptor 71 along the aligning section (not shown). At this time, the insertion portion side electrode terminal 83 on the anode side of the pair of insertion portion side electrode terminals 83 and 85 is not yet in contact with the adaptor side electrode terminal 30 of the pair of adaptor side electrode terminals (only the anode side is shown in the diagram).

The male screw section 33 is screwed into the female screw section 22 of the connection ring 21 of the adaptor 71 to bring the distal end surface 212a of the cylinder section 212 in contact with the bottom section 76 of the depressed section 75 of the lens cylinder section 73, and air from an air source (not shown) is introduced into the air tube 216 and is supplied into the balloon 215.

At this time, as shown in FIG. 17B to FIG. 17D, the balloon 215 begins to expand outward in the radial direction, and the distal end section of the insertion portion side flexible substrate 82 mounted on the balloon 215 bends as a curved surface along the expanded surface of the balloon 215. Then the insertion portion side electrode terminal 83 moves outward in the radial direction to press the adaptor side electrode terminal 30 outward in the radial direction. As a result, the area of contact between the insertion portion side electrode terminal 83 and the adaptor side electrode terminal 30 successively increases.

Thus, the pair of adaptor side electrode terminals and the pair of insertion portion side electrode terminals are respectively conducted with each other and the adaptor 71 and the main body distal end section 211 are electrically connected.

According to the endoscope 210, an effect similar to that of the other embodiments in which the electrode terminals are biased in the radial direction by a spring member can be achieved. In particular, since a biasing force is given to the balloon 215 by supplying it with air, the performance of the biasing device can be maintained for a longer period of time compared to the case of providing a constant biasing device.

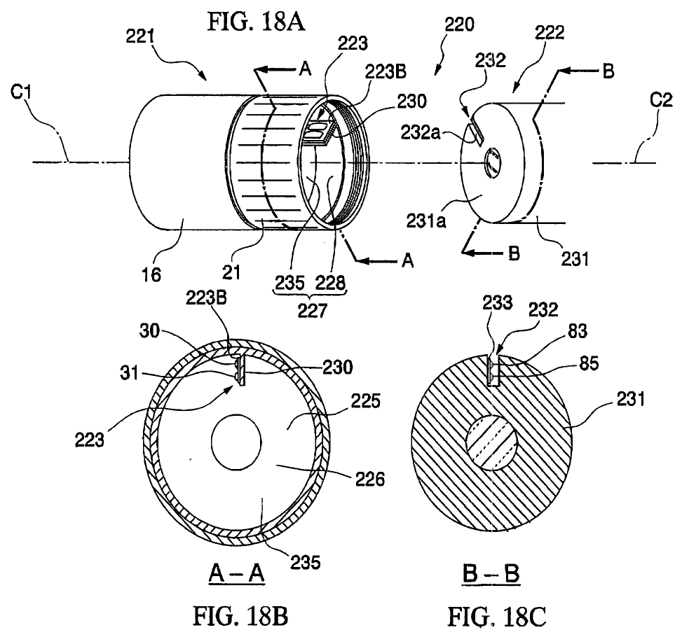

Next, a fourteenth embodiment is described, with reference to FIG. 18A to 18C.

Components similar to those in the other embodiments described above are given the same reference symbols, and descriptions thereof are omitted.

The fourteenth embodiment differs from the third embodiment in that a pair of adaptor side electrode terminals 30 and 31 in an adaptor 221 of an endoscope 220 according to the present embodiment, is arranged side by side in the radial direction from the outer circumferential surface of the adaptor 221 towards the central axis C1 as shown in FIGS. 18A and 18B, and a pair of insertion portion side electrode terminals 83 and 85 arranged on a main body distal end section 222 are also arranged side by side in the same direction.

That is, an extension section 223B of a flexible substrate 223 is formed so as to project from a disc section (not shown) towards the proximal end side taking the radial direction of the disc section as a crosswise direction. Then, on side surfaces of an aluminum substrate (not shown) and a base section 226 of a lens cylinder section 225, instead of a through hole, a slit (not shown) is arranged, that respectively engages with the extension section 223B to stop the extension section 223B from projecting from the outer surface when arranged so that the crosswise direction of the extension section 223B substantially matches the radial direction of the side surfaces of the aluminum substrate and the base section 226 of the lens cylinder section 225.

A support section 230 that projects inward in the radial direction to support the extension section 223B projecting from the slit, is arranged on a side surface section 228 of a depressed section 227 of the lens cylinder section 225. At this time, the adaptor side facing surfaces 32 of the pair of adaptor side electrode terminals 30 and 31 are arranged to face in the circumferential direction of the lens cylinder section 225.

As shown in FIG. 18C, an engaging groove 232 into which the support section 230 can be inserted, is formed on the side surface of the cylinder section 231 at the main body distal end section 222, from the side surface towards the inside in the radial direction. Then, when the adaptor 221 is attached to the main body distal end section 222, the insertion portion side flexible site 233 is arranged on a side surface 232a of the engaging groove 232 so that the pair of adaptor side electrode terminals 30 and 31 faces the pair of insertion portion side electrode terminals 83 and 85.

Here, the width of the engaging groove 232 is sufficient for the adaptor side facing surfaces 32 of the pair of adaptor side electrode terminals 30 and 31 and the insertion portion side facing surfaces 47 of the pair of insertion portion side electrode terminals 83 and 85 to come into contact with each other when the adaptor 221 is attached with the pair of insertion portion side electrode terminals 83 and 85 arranged on the side surface 232a.

The endoscope 220 is not comprising the aligning section seen in the respective embodiments mentioned above, and the support section 230 has a function as an engaging section, and the engaging groove 232 has a function as a groove section.

Next, the procedures and effects when using the endoscope 220 according to the present embodiment is described.

First, with the support section 230 of the adaptor 221 being engaged in the engaging groove 232 of the main body distal end section 222, the cylinder section 231 of the main body distal end section 222 is inserted into the connection ring 21.

At this time, the adaptor side facing surface and the insertion portion side facing surface begin to come in contact with each other, and the area of their contact increases with insertion as they gradually come into contact in the attachment direction.

Then, the distal end surface 231a of the cylinder section 231 and the bottom section 235 of the depressed section 227 of the lens cylinder section 225 are brought into contact with each other by screwing the male screw section 33 and the female screw section 22 together.

At this time, the area of contact between the adaptor side facing surface 32 and the insertion portion side facing surface 47 reaches a maximum, and the pair of adaptor side electrode terminals 30 and 31 and the pair of insertion portion side electrode terminals 83 and 85 are conducted, respectively, and the adaptor and the main body distal end section 222 are electrically connected.

According to the endoscope 220, the pair of adaptor side electrode terminals 30 and 31 and the pair of insertion portion side electrode terminals 83 and 85 can be brought into surface contact on their respective facing surfaces, and the area of their contact can be successively increased from the start to the end of attachment. Therefore, the adaptor 221 and the main body distal end section 222 can be electrically connected more reliably than in the case of line contact.

Moreover, since an aligning section is not provided, the number of parts can be reduced and assembly can be improved.

The technical range of the present invention is not limited to the embodiments mentioned above, and various modifications may be made without departing the scope of the invention.

For example, in the third embodiment described above, when the extension section 72A of the flexible substrate 72 is pressed outward in the radial direction, the extension section 72A moves in the radial direction within the gap 78A formed between the extension section 72A and the side sake section 77 of the depressed section 75 of the lens cylinder section 73. However, in the case where the extension section is formed from a soft member, the gap may be filled in with an adhesive agent.

In this case, since one portion part way along the extension section bends, instead of the end section of the extension section moving in the radial direction when attached, the degree of this bend adjusts the pressure of contact between the pair of adaptor side electrode terminals and the pair of the insertion portion side electrode terminals, and an effect similar to that of the above embodiments can be achieved.

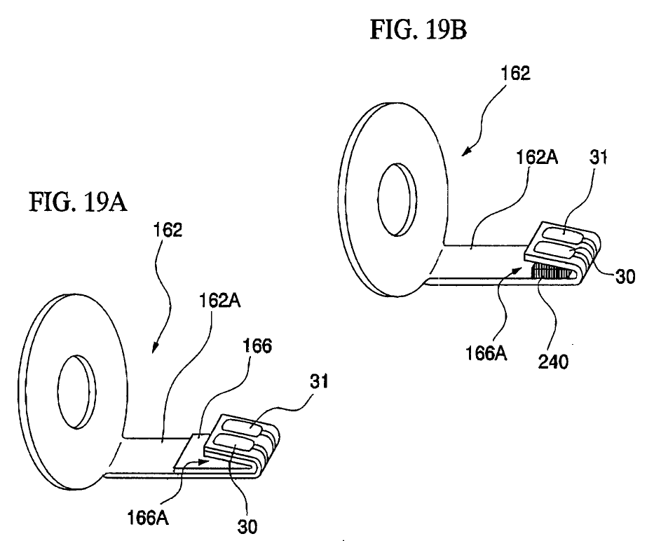

Moreover, in the space section 166A of the plate spring section 166 arranged on the flexible substrate 162 according to the above ninth embodiment shown in FIG. 19A, as shown in FIG. 19B, an elastic body (biasing device) 240 may be arranged, angled at a constant angle.

In this case, the pair of insertion portion side electrode terminals (not shown) can be pressed by an elastic force that occurs when the elastic body 240 deforms with deformation of the plate spring section 166, and the pair of adaptor side electrode terminals and the pair of insertion portion side electrode terminals can be brought into contact with each other more reliably as a result.

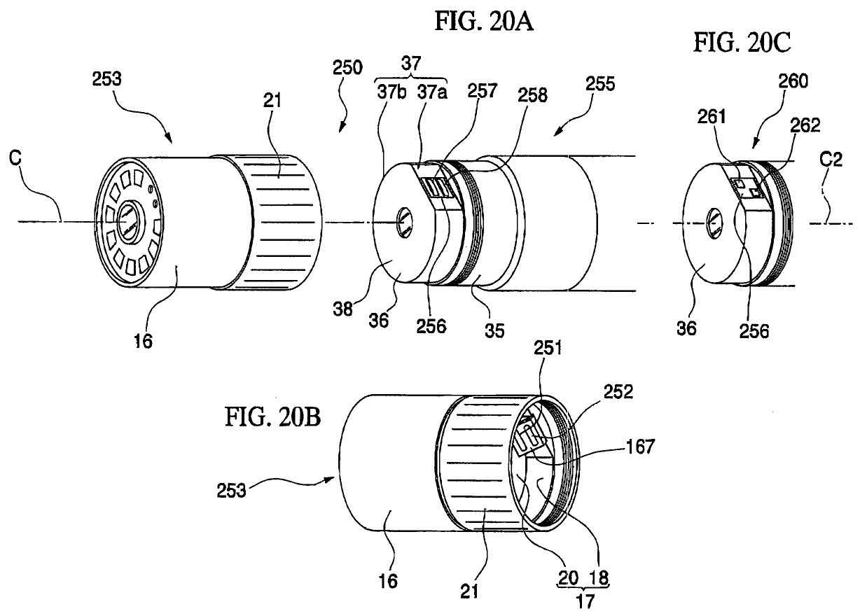

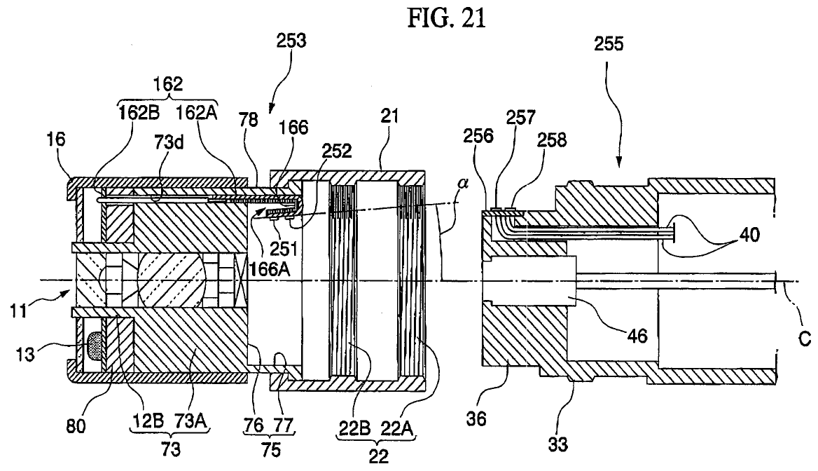

Furthermore, in the above embodiments, the pair of adaptor side electrode terminals and the pair of insertion portion side electrode terminals are respectively arranged in pairs in a direction orthogonal to the direction in which the adaptor and the insertion portion are attached to each other. However, arrangement of the electrode terminals is not limited to the direction described above, and it may be a direction substantially the same as the direction in which the adaptor and the insertion portion are attached to each other. For example, as shown in FIG. 20A, FIG. 20B and FIG. 21, the pair of adaptor side electrode terminals 251 and 252 of an endoscope 250 may be arranged on the flexible substrate 162 according to the above ninth embodiment along a direction orthogonal to the central axis C, which is a direction in which an adaptor 253 and a main body distal end section 255 are attached to each other. In this case, on the main body distal end section 255, a pair of insertion portion side electrode terminals 257 and 258 is arranged on one insertion portion side terminal substrate 256 arranged on the second plane surface of the protruding section 36 so as to respectively face the pair of insertion portion side electrode terminals 251 and 252.

Moreover, as shown in FIG. 20C, a pair of insertion portion side electrode terminals 261 and 262 of a main body distal end section 260 may be arranged in a line in a direction diagonal to the central axis C2. In this case, the pair of adaptor side electrode terminals (not shown) is also arranged diagonally so as to face the pair of insertion portion side electrode terminals 261 and 262.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An endoscope comprising:
an insertion portion formed in an elongated cylindrical shape;
a cylindrical shaped image pickup adaptor, the pickup adaptor comprising, an electric device arranged on the distal end side thereof, and a proximal end side of the image pickup adaptor being attached to and removed from the distal end side of the insertion portion, wherein a pair of adaptor side electrode terminals conducting with the electric device are arranged on the image pickup adaptor; and
a pair of insertion portion side electrode terminals that respectively come into contact with and are electrically connected to the pair of adaptor side electrode terminals, are arranged on the insertion portion so as to respectively face, and be able to come in contact with, the pair of adaptor side electrode terminals, when the image pickup adaptor is attached to the insertion portion.

2. An endoscope according to claim 1, wherein the pair of adaptor side electrode terminals and the pair of insertion portion side electrode terminals are arranged in pairs in a direction different from the direction in which the image pickup adaptor and the insertion portion are attached and detached to and from each other.

3. An endoscope according to claim 1, wherein the pair of adaptor side electrode terminals and the pair of insertion portion side electrode terminals are arranged so as to face each other in a direction orthogonal to the direction in which the image pickup adaptor and the insertion portion are attached and detached to and from each other.

4. An endoscope according to claim 1, wherein the pair of adaptor side electrode terminals and the pair of insertion portion side electrode terminals are arranged so as to face each other in a direction inclined at a constant angle to the direction in which the image pickup adaptor and the insertion portion are attached detached to and from each other.

5. An endoscope according to claim 1, further comprising an aligning section which adjusts the orientation of the image pickup adaptor to the direction the pair of adaptor side electrode terminals and the pair of insertion portion side electrode terminals facing each other, when the image pickup adaptor is attached to the insertion portion.

6. An endoscope according to claim 1, further comprising a biasing device which adjusts a pressing force of the pair of adaptor side electrode terminals and the pair of insertion portion side electrode terminals, when the image pickup adaptor is attached to the insertion portion.

7. An endoscope according to claim 6, wherein the biasing device is a spring member.

8. An endoscope according to claim 6, wherein the biasing device is a rubber member.

9. An endoscope according to claim 8, wherein the rubber member has a conducting property in a direction of pressing.

10. An endoscope according to claim 6, wherein the biasing device is a balloon that inflates when supplied with fluid.

11. An endoscope according to claim 1, wherein at least one of the pair of iron portion side electrode terminals and the pair of adaptor side electrode terminals is formed from an elastic member.

12. An endoscope according to claim 1, wherein at least one of the pair of insertion portion side electrode terminals and the pair of adaptor side electrode terminals is arranged on a flexible substrate that can bend in a plate-thickness direction.

13. An endoscope according to claim 12, wherein the flexible substrate is formed in a strip shape.

14. An endoscope according to claim 12, wherein the flexible substrate is formed in a cylindrical shape.

15. An endoscope according to claim 1, wherein the pair of insertion portion side electrode terminals and the pair of adaptor side electrode terminals are respectively comprising facing surfaces that face each other.

16. An endoscope comprising:
an insertion portion formed in an elongated cylindrical shape;
a cylindrical shaped image pickup adaptor, the pickup adaptor comprising, an electric device arranged on the distal end side thereof, and a proximal end side of the image pickup adaptor being attached to and removed from the distal end side of the insertion portion, wherein a pair of adaptor side electrode terminals conducting with the electric device are arranged on the image pickup adaptor; and
a pair of insertion portion side electrode terminals that respectively come into contact with and are electrically connected to the pair of adaptor side electrode terminals, are arranged on the insertion portion so as to respectively face, and be able to come in contact with, the pair of adaptor side electrode terminals, when the image pickup adaptor is attached to the insertion portion,
wherein the pair of adaptor side electrode terminals and the pair of insertion portion side electrode terminals are arranged in pairs in a direction different from the direction in which the image pickup adaptor and the insertion portion are attached and detached to and from each other.

17. An endoscope according to claim 16, wherein the biasing device is a rubber member and has a conducting property in a direction of pressing.

18. An endoscope comprising:
an insertion portion formed in an elongated cylindrical shape;
a cylindrical shaped image pickup adaptor, the pickup adaptor comprising, an electric device arranged on the distal end side thereof, and a proximal end side of the image pickup adaptor being attached to and removed from the distal end side of the insertion portion, wherein a pair of adaptor side electrode terminals conducting with the electric device are arranged on the image pickup adaptor; and
a pair of insertion portion side electrode terminals that respectively come into contact with and are electrically connected to the pair of adaptor side electrode terminals, are arranged on the insertion portion so as to respectively face, and be able to come in contact with, the pair of adaptor side electrode terminals, when the image pickup adaptor is attached to the insertion portion,
wherein the pair of adaptor side electrode terminals and the pair of insertion portion side electrode terminals are arranged in pairs in a direction different from the direction in which the image pickup adaptor and the insertion portion are attached and detached to and from each other, and
at least one of the pair of insertion portion side electrode terminals and the pair of adaptor side electrode terminals is arranged on a flexible substrate that can bend in a plate-thickness direction.

19. An endoscope according to claim 18, wherein the flexible substrate is formed in a strip shape.

20. An endoscope according to claim 18, wherein the flexible substrate is formed in a cylindrical shape.
